SEQUENCE LISTING (46 sequences)

SEQ ID NO: 1 - protein, 185 aa, Artificial
  GLuc mutant(I90L) version 1.0
MGVKVLFALICIAVAEAKPTENNEDFNIVAVASNFATTDLDADRGKLPGKKLPLEVLKEMEANARKAGCTRGCLICLSHIKCTPKMKKFLPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 2 - protein, 189 aa, Artificial
  GLuc mutant(I90L) version 1.1 (+KDEL)
MGVKVLFALICIAVAEAKPTENNEDFNIVAVASNFATTDLDADRGKLPGKKLPLEVLKEMEANARKAGCTRGCLICLSHIKCTPKMKKFLPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 3 - protein, 170 aa, Artificial
  GLuc mutant (I90L) version 1.2 (-Signal peptide)
MGKPTENNEDFNIVAVASNFATTDLDADRGKLPGKKLPLEVLKEMEANARKAGCTRGCLICLSHIKCTPKMKKFLPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 4 - protein, 174 aa, Artificial
  GLuc mutant (I90L) version 1.3 (-Signal peptide, +KDEL)
MGKPTENNEDFNIVAVASNFATTDLDADRGKLPGKKLPLEVLKEMEANARKAGCTRGCLICLSHIKCTPKMKKFLPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 5 - protein, 6 aa, Artificial
  His-Tag
HHHHHH

SEQ ID NO: 6 - protein, 8 aa, Artificial
  FLAG-Tag (epitope)
DYKDDDDK

SEQ ID NO: 7 - protein, 10 aa, Artificial
  Myc-Tag (epitope)
EQKLISEEDL

SEQ ID NO: 8 - protein, 9 aa, Artificial
  HA-Tag (epitope)
YPYDVPDYA

SEQ ID NO: 9 - protein, 14 aa, Artificial
  V5-Tag (epitope)
GKPIPNPLLGLDST

SEQ ID NO: 10 - protein, 11 aa, Artificial
  T7-Tag (epitope)
MASMTGGQQMG

SEQ ID NO: 11 - protein, 11 aa, Artificial
  Membrane-localization signal
MLCCMRRTKQV

SEQ ID NO: 12 - protein, 10 aa, Artificial
  Cytosol-localization signal
GRKKRRQRRR

SEQ ID NO: 13 - protein, 8 aa, Artificial
  Nucleus-localization signal
DPKKKRKV

SEQ ID NO: 14 - protein, 4 aa, Artificial
  ER-localization signal
KDEL

SEQ ID NO: 15 - protein, 189 aa, Artificial
  GLuc mutant (F89W)
MGVKVLFALICIAVAEAKPTENNEDFNIVAVASNFATTDLDADRGKLPGKKLPLEVLKEMEANARKAGCTRGCLICLSHIKCTPKMKKWIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 16 - protein, 189 aa, Artificial
  GLuc mutant (H95E)
MGVKVLFALICIAVAEAKPTENNEDFNIVAVASNFATTDLDADRGKLPGKKLPLEVLKEMEANARKAGCTRGCLICLSHIKCTPKMKKFIPGRCETYEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 17 - protein, 189 aa, Artificial
  GLuc mutant (Y97W)
MGVKVLFALICIAVAEAKPTENNEDFNIVAVASNFATTDLDADRGKLPGKKLPLEVLKEMEANARKAGCTRGCLICLSHIKCTPKMKKFIPGRCHTWEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 18 - protein, 189 aa, Artificial
  GLuc mutant (F89W/I90L); named 8990
MGVKVLFALICIAVAEAKPTENNEDFNIVAVASNFATTDLDADRGKLPGKKLPLEVLKEMEANARKAGCTRGCLICLSHIKCTPKMKKWLPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 19 - protein, 189 aa, Artificial
  GLuc mutant (I90L/I115L)
MGVKVLFALICIAVAEAKPTENNEDFNIVAVASNFATTDLDADRGKLPGKKLPLEVLKEMEANARKAGCTRGCLICLSHIKCTPKMKKFLPGRCHTYEGDKESAQGGIGEAIVDLPEIPGFKDLEPMEQFIAQVDLCVDCTTGCLKGLANVQCSDLLKKWLPQRCATFASKIQGQVDKIKGAGGDKDEL

SEQ ID NO: 20 - DNA, 570 bp, Artificial
  A human codon-optimized GLuc variant cDNA (F89W/I90L/H95E/Y97W); Mon3
atgggcgtgaaggtcctgttcgccctgatctgcatcgccgtggccgaggccaagcccaccgagaacaacgaggacttcaacattgtggccgtggccagcaactttgcgaccacggacctcgacgccgatcgcgggaagttgcccggaaagaagctgccgctggaggtcctcaaggagatggaggccaacgcccggaaggccggctgcaccaggggatgcctgatctgcctgtcccacattaagtgcacgcccaagatgaagaagtggctccccggccgctgcgagacctgggagggagacaaggagtccgcccagggcggtatcggagaggcgattgtcgatatcccggagataccggggtttaaggatttggagcccatggagcagttcatcgcgcaggtcgatctttgcgtggactgcacgacgggctgcctcaaggggctcgccaacgtgcagtgctccgacctgctcaagaagtggctaccgcagcgctgcgcgaccttcgccagcaagattcagggacaggtggacaagatcaagggggccggcggatccaaggacgagctgtga

SEQ ID NO: 21 - protein, 189 aa, Artificial
  GLuc mutant (H95Q/D100N)
MGVKVLFALICIAVAEAKPTENNEDFNIVAVASNFATTDLDADRGKLPGKKLPLEVLKEMEANARKAGCTRGCLICLSHIKCTPKMKKFIPGRCQTYEGNKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 22 - protein, 189 aa, Artificial
  GLuc mutant (I90V)
MGVKVLFALICIAVAEAKPTENNEDFNIVAVASNFATTDLDADRGKLPGKKLPLEVLKEMEANARKAGCTRGCLICLSHIKCTPKMKKFVPGRCHTYEGNKESAQGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 23 - protein, 189 aa, Artificial
  GLuc mutant (I108V)
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGNKESAQGGVGEAIVDIPEIPGFKDLEPMEQFIAQVDLCVDCTTGCLKGLANVQCSDLLKKWLPQRCATFASKIQGQVDKIKGAGGDKDEL

SEQ ID NO: 24 - protein, 189 aa, Artificial
  GLuc mutant (I118V)
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGNKESAQGGIGEAIVDIPEVPGFKDLEPMEQFIAQVDLCVDCTTGCLKGLANVQCSDLLKKWLPQRCATFASKIQGQVDKIKGAGGDKDEL

SEQ ID NO: 25 - protein, 189 aa, Artificial
  GLuc mutant (I90L/I115V)
MGVKVLFALICIAVAEAKPTENNEDFNIVAVASNFATTDLDADRGKLPGKKLPLEVLKEMEANARKAGCTRGCLICLSHIKCTPKMKKFLPGRCHTYEGNKESAQGGIGEAIVDVPEIPGFKDLEPMEQFIAQVDLCVDCTTGCLKGLANVQCSDLLKKWLPQRCATFASKIQGQVDKIKGAGGDKDEL

SEQ ID NO: 26 - protein, 214 aa, Artificial
  MpLuc1 mutant (Y113F)
MMEIQVLFALICFALVQANPTENKDDIDIVGVEGKFGTTDLETDLFTIVEDMNVISRDTNLANSDADRGKMPGKKLPLEVLIEMEANARKAGCTRGCLICLSKIKCTAKMKVFIPGRCHDYGGDKKTGQAGIVGAIVDIPEISGFKELGPMEQFIAQVDLCADCTTGCLKGLANVKCSALLKKWLPDRCASFADKIQSEVDNIKGLAGDRKDEL

SEQ ID NO: 27 - protein, 214 aa, Artificial
  MpLuc1 mutant (I114L)
MMEIQVLFALICFALVQANPTENKDDIDIVGVEGKFGTTDLETDLFTIVEDMNVISRDTNLANSDADRGKMPGKKLPLEVLIEMEANARKAGCTRGCLICLSKIKCTAKMKVYLPGRCHDYGGDKKTGQAGIVGAIVDIPEISGFKELGPMEQFIAQVDLCADCTTGCLKGLANVKCSALLKKWLPDRCASFADKIQSEVDNIKGLAGDRKDEL

SEQ ID NO: 28 - protein, 210 aa, Artificial
  MpLuc1 mutant (Y113W/I114L/H119E/Y121W); MpLuc4
MMEIQVLFALICFALVQANPTENKDDIDIVGVEGKFGTTDLETDLFTIVEDMNVISRDTNLANSDADRGKMPGKKLPLEVLIEMEANARKAGCTRGCLICLSKIKCTAKMKVWLPGRCEDWGGDKKTGQAGIVGAIVDIPEISGFKELGPMEQFIAQVDLCADCTTGCLKGLANVKCSALLKKWLPDRCASFADKIQSEVDNIKGLAGDR

SEQ ID NO: 29 - protein, 219 aa, Artificial
  MLuc mutant (I123L)
MDIKVVFTLVFSALVQAKSTEFDPNIDIVGLEGKFGITNLETDLFTIWETMEVMIKADIADTDRASNFVATETDANRGKMPGKKLPLAVIMEMEANAFKAGCTRGCLICLSKIKCTAKMKVYLPGRCHDYGGDKKTGQAGIVGAIVDIPEISGFKEMAPMEQFIAQVDRCASCTTGCLKGLANVKCSELLKKWLPDRCASFADKIQKEVHNIKGMAGDR

SEQ ID NO: 30 - protein, 219 aa, Artificial
  MLuc mutant (Y122W/I123L/H128E/Y130W); MLuc4
MDIKVVFTLVFSALVQAKSTEFDPNIDIVGLEGKFGITNLETDLFTIWETMEVMIKADIADTDRASNFVATETDANRGKMPGKKLPLAVIMEMEANAFKAGCTRGCLICLSKIKCTAKMKVWLPGRCEDWGGDKKTGQAGIVGAIVDIPEISGFKEMAPMEQFIAQVDRCASCTTGCLKGLANVKCSELLKKWLPDRCASFADKIQKEVHNIKGMAGDR

SEQ ID NO: 31 - protein, 195 aa, Artificial
  Obelia luciferase mutant (L95I/A96L/K101E/W130L); OLuc4
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQILTSELEKLARNEPTLIREWGDAVFDIFDKDGSGTITLDEWKAYGKISGISPSQEDCEATFRHCDLDNSGDLDVDEMTRQHLGFWYTLDPEADGLYGNGVP

SEQ ID NO: 32 - protein, 196 aa, Artificial
  aqualine luciferase mutant (L96I/A97L/E102H/Y104W); ALuc4
MTSKQYSVKLTSDFDNPRWIGRHKHMFNFLDVNHNGKISLDEMVYKASDIVINNLGATPEQAKRHKDAVEAFFGGAGMKYGVETDWPAYIEGWKKILTDELHKWAKNEPTLIRIWGDALFDIVDKDQNGAITLDEWKAYTKAAGIIQSSEDWEETFRVCDIDESGQLDVDEMTRQHLGFWYTMDPACEKLYGGAVP

SEQ ID NO: 33 - protein, 189 aa, Artificial
  MpLuc2 luciferase mutant (F92W/I93L/H98E/Y100W); MpLuc5
MGVKLIFAVLCVAAAQAATINENFEDIDVVAIGGSFALDVDANRGGHGGHPGKKMPKEVLVEMEANAKRAGCHRGCLICLSHIKCTKKMKKWLPGRCESWEGDKDSAQGGIGEEIVDMPEIPGFKDKEPMDQFIAQVDLCVDCTTGCLKGLANVHCSALLKKWLPSRCKTFASKIQSQVDTIKGLAGDR

SEQ ID NO: 34 - protein, 196 aa, Artificial
  Oplophorus luciferase mutant (I103L/F104W/Y108W/V110L); OpLuc4
MAYSTLFIIALTAVVTQASSTQKSNLTFTLADFVGDWQQTAGYNQDQVLEQGGLSSLFQALGVSVTPIQKVVLSGENGLKADIHVIIPYEGLSGFQMGLIEMLWKVVWPLDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 35 - protein, 198 aa, Artificial
  Pleuromamma luciferase mutant (T101W/W102L/E109H/D111W); PLuc4
MLRNCARKQEQVCADVTEMKCQAVAWADCGPRFDSTGRNRCQVQYKDYAYKSCVEVDYTVPHRKQVPECKQVTKDNCVTDWEVDANGNKVWGGTEKCTPVWLEECNIVHKWVDFPTVKTECGILSHLKYADFIEGPSHSLSMRTNCQVKSSLDCRPVKTRKCATVEYHECSMKPQEDCSPVTVHIPDQEKVHQKKCLT

SEQ ID NO: 36 - protein, 311 aa, Artificial
  Renilla luciferase mutant (F105W/E106L/Y129L/Y131W); RLuc4
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWWLLINIPKKIIFVGHDWGACLAFHLSWEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 37 - DNA, 660 bp, Artificial
  A human codon-optimized MLuc variant cDNA; 4 mutated
atggacataaaggtggtgtttacgctggtatttagcgcactggttcaggccaaatcgacggagtttgatccgaatattgacattgttggattggaaggcaaattcggcattaccaacctggaaaccgatctgttcaccatctgggaaacgatggaggtcatgatcaaagccgacattgccgataccgatcgggcttcgaactttgttgctacagaaacggatgcaaatcgcgggaaaatgccgggcaagaaactgccgttagcggtaatcatggaaatggaagcgaacgcgttcaaagcgggttgcacacgtgggtgtctgatctgcttgagcaagatcaaatgcacggccaaaatgaaagtctggttaccaggacgttgcgaagattggggtggcgacaagaaaaccggtcaagcaggcattgtgggtgcgattgtggatatccccgaaatttctggcttcaaagagatggcgccgatggaacagttcattgcccaagttgaccgctgtgcgagttgcactactggctgtctgaaagggcttgcgaatgtcaaatgctcagaactgctcaagaaatggctccctgatcgttgtgcctcctttgcagacaaaatccagaaagaggtgcataacatcaaaggtatggctggtgatcgctaa

SEQ ID NO: 38 - DNA, 633 bp, Artificial
  human codon-optimized MpLuc1 cDNA; 4-mutated
atgatggaaatacaagttctttttgctctcatttgctttgcattggtgcaggccaatcccactgagaataaggacgacatcgacatcgttggagtcgaaggcaagtttgggaccactgatctggaaacagacctgttcaccatcgttgaggatatgaacgtgatttcccgagacacgaatctggcgaactctgacgcagatagagggaagatgccaggtaagaaactccctctggaagtgttgatagagatggaggctaatgcccggaaagcaggatgcacaagaggctgcctgatttgcctcagcaaaatcaagtgtacagccaagatgaaagtctggctgcccggccgttgcgaggattggggcggtgacaagaagaccggacaagccgggatagtaggtgccatcgtggatatcccggagatttccgggttcaaagagctgggacctatggaacagttcattgcgcaggtggacttgtgtgccgactgtaccactggctgtctgaaaggccttgcaaacgtgaaatgctcagctctgctcaagaaatggctgccagatcgctgtgccagctttgccgacaagatccagagtgaagtggacaacatcaagggacttgctggggataggtga

SEQ ID NO: 39 - DNA, 582 bp, Artificial
  human codon-optimized MpLuc1 cDNA; 1-mutated variant without a secretion peptide
atgggccccactgagaataaggacgacatcgacatcgttggagtcgaaggcaagtttgggaccactgatctggaaacagacctgttcaccatcgttgaggatatgaacgtgatttcccgagacacgaatctggcgaactctgacgcagatagagggaagatgccaggtaagaaactccctctggaagtgttgatagagatggaggctaatgcccggaaagcaggatgcacaagaggctgcctgatttgcctcagcaaaatcaagtgtacagccaagatgaaagtctacctgcccggccgttgccacgattatggcggtgacaagaagaccggacaagccgggatagtaggtgccatcgtggatatcccggagatttccgggttcaaagagctgggacctatggaacagttcattgcgcaggtggacttgtgtgccgactgtaccactggctgtctgaaaggccttgcaaacgtgaaatgctcagctctgctcaagaaatggctgccagatcgctgtgccagctttgccgacaagatccagagtgaagtggacaacatcaagggacttgctggggataggtga

SEQ ID NO: 40 - protein, 494 aa, Artificial
  cPresso; an integrated molecule-format probe carrying circularly permutated GLuc.
MNALLRYLLDKDGSGGGGIGEAIVDIPEIPGFKDLEPMEQFIAQVDLCVDCTTGCLKGLANVQCSDLLKKWLPQRCATFASKIQGQVDKIKGAGGDKDELGGSGRGGMKPTENNEDFNIVAVASNFATTDLDADRGKLPGKKLPLEVLKEMEANARKAGCTRGCLICLSHIKCTPKMKKFIPGRCHTYEGDKESAQGGGGGTMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 41 - protein, 506 aa, Artificial
  cPressoMAX; an integrated molecule-format bioluminescent probe carrying a superluminescent GLuc mutant.
MEQKLISEEDLHMNALLRYLLDKDLKGGGGIGEAIVDIPEIPGFKDLEPMEQFIAQVDLCVDCTTGCLKGLANVQCSDLLKKWLPQRCATFASKIQGQVDKIKGAGGDKDELGDIGRGGMKPTENNEDFNIVAVASNFATTDLDADRGKLPGKKLPLEVLKEMEANARKAGCTRGCLICLSHIKCTPKMKKWLPGRCHTYEGDKESAQGGGGGTMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 42 - protein, 453 aa, Artificial
  SimGR3; an integrated molecule-format probe carrying an LXXLL motif and GLuc.
MKPTENNEDFNIVAVASNFATTDLDADRGKLPGKKLPLEVLKEMEANARKAGCTRGCLICLSHIKCTPKMKKFIPGRCHTYEGDKESAQGGGGGTQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQKVDGGNGGRNALLRYLLDKDGSGGGGIGEAIVDIPEIPGFKDLEPMEQFIAQVDLCVDCTTGCLKGLANVQCSDLLKKWLPQRCATFASKIQGQVDKIKGAGGDKDEL

SEQ ID NO: 43 - protein, 449 aa, Artificial
  8990N; an integrated molecule-format probe carrying 8990.
MKPTENNEDFNIVAVASNFATTDLDADRGKLPGKKLPLEVLKEMEANARKAGCTRGCLICLSHIKCTPKMKKWLPGRCHTYEGDKESAQGGGGGTQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQKVDGGNGGRNALLRYLLDKDGSGGGGIGEAIVDIPEIPGFKDLEPMEQFIAQVDLCVDCTTGCLKGLANVQCSDLLKKWLPQRCATFASKIQGQVDKIKGAGGD

SEQ ID NO: 44 - protein, 449 aa, Artificial
  Mon3N; an integrated molecule-format probe carrying Mon3.
MKPTENNEDFNIVAVASNFATTDLDADRGKLPGKKLPLEVLKEMEANARKAGCTRGCLICLSHIKCTPKMKKWLPGRCETWEGDKESAQGGGGGTQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQKVDGGNGGRNALLRYLLDKDGSGGGGIGEAIVDIPEIPGFKDLEPMEQFIAQVDLCVDCTTGCLKGLANVQCSDLLKKWLPQRCATFASKIQGQVDKIKGAGGD

SEQ ID NO: 45 - protein, 261 aa, Artificial
  GAL4-linked Src SH2 cDNA for Two hybrid assays
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFPGIRRLVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRL

SEQ ID NO: 46 - protein, 299 aa, Artificial
  VP16-linked ER LBD cDNA for Two hybrid assays
MTAPPTDVSLGDELHLDGEDVAMAHADALDDFDLDMLGDGDSPGPGSPGIRRLSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLH

(19) United States
(12) Patent Application Publication
(10) Pub. No.: US 2012/0034672 A1
(43) Pub. Date: Feb. 9, 2012

(54) SUPERLUMINESCENT LUCIFERASE VARIANT WITH PROLONGED BIOLUMINESCENCE

(76) Inventors: SungBae KIM, Tsukuba-shi (JP); Hiroaki Tao, Tsukuba-shi (JP); Moritoshi Sato, Bunkyo-ku (JP)

(21) Appl. No.: 13/274,764

(22) Filed: Oct. 17, 2011

Related U.S. Application Data
(63) Continuation-in-part of application No. PCT/JP2010/052511, filed on Feb. 19, 2010.

(30) Foreign Application Priority Data
Apr. 17, 2009 (JP) 2009-101025

(51) Int. Cl. C12N 9/02; C12N 1/19; C12N 1/21; C12N 5/10; C12N 15/53; C12N 15/63

(52) U.S. Cl. 435/189; 536/23.2; 435/320.1; 435/252.33; 435/348; 435/365; 435/358; 435/367; 435/369; 435/254.2

(57) ABSTRACT

This invention provides a genetically modified marine luciferase such as Gaussia luciferase, which has high bioluminescence intensity, and has high bioluminescence stability and/or red-shifted wavelength. Specifically disclosed is a luciferase variant with improved optical property obtained by replacing at least one amino acid residue among the amino acid sequence of a marine luciferase at positions corresponding to positions 89 to 118 in the amino acid sequence of Gaussia luciferase (GLuc), wherein an amino acid residue at a position corresponding to at least one selected from positions 89, 90, 95, 97, 100, 108, 112, 115, and 118 in the amino acid sequence of GLuc is replaced by way of conservative amino acid replacement. The above-mentioned replacement in a marine luciferase improves enzymatic activity of the luciferase. Also disclosed is a bioluminescent probe having an improved optical property, which is produced using the luciferase variant of the present invention.

### CROSS-REFERENCE TO RELATED APPLICATIONS

This application claims priority to the specification of JP Application No. 2009-101025, filed on Apr. 17, 2009, and the specification of PCT International Application PCT/JP2010-052511, filed on Feb. 19, 2010, the disclosure of which is incorporated herein by reference in its entirety.

### TECHNICAL FIELD

The present invention relates to the establishment of a method for improving the function, such as optical properties, and luminescence stability of a luciferase by way of genetic modification of marine luciferases (i.e. a genetically modified marine luciferase), and also relates to a stable artificial luciferase having super high bioluminescence synthesized by the method (i.e. a superluminescent luciferase with a prolonged bioluminescence, which are artificially synthesized).

### BACKGROUND ART

Many molecular phenomena occurring in cells or cell-free systems in nature (for example, conformation changes and phosphorylation of proteins, protein-protein interactions, and production of second messengers) are highly important signatures for us to understand the molecular nature and to explore the molecular mechanisms dominating life phenomena. Presently, various methods, including (i) FRET (Non-patent Literature 1 and 2), (ii) BRET (Non-patent Literature 3), and (iii) protein-fragment complementation assay (Non-patent Literature 4 and 5), have been developed to measure such molecular phenomena.

In recent years, the present inventors have conducted research and development regarding bioluminescence imaging using unique molecular design technology. More specifically, the inventors measured translocation of transcription factors into the nucleus or nongenomic protein-protein interactions in the cytosol using protein splicing (Non-patent Literature 6 and 7). Further, the inventors developed a single molecule-format bioluminescent probe in which all of the necessary elements for signal recognition and bioluminescence emission are integrated (Non-patent Literature 8 and 9). Thereafter, the probes were multicolorized and applied to simultaneously image multiple signal-transduction processes (Non-patent Literature 10). Moreover, the inventors further developed a circular permutation technique (Non-patent Literature 11), or a molecular design technology of small luciferases (Non-patent Literature 12), as strategies for improving the ligand sensitivity of the bioluminescent probe itself. These technologies have been used as means for efficiently measuring molecular phenomena in cellular and cell-free systems.

Fluorescence imaging is more widespread than luminescence imaging as a main research tool for exploring intra- or extracellular molecular phenomena. However, fluorescent proteins company autofluorescence, and therefore generate a high background, requiring an external light source. Therefore, fluorescence imaging requires a big instrumentation such as a fluorescence microscope and a sophisticated light-filtering system. Fluorescence imaging also has a drawback in that maturation of fluorescence chromophore takes at least several hours to several days. Further, when using a fluorescence microscope, the observable cell numbers for each measurement are limited, which has hampered quantitative analysis (Non-patent Literature 6).

On the other hand, the bioluminescence imaging using a luciferase has, despite the many advantages, a critical problem of poor bioluminescence intensities of luciferases, which results in hampering popular use of bioluminescence imaging compared to fluorescence imaging. Because the bioluminescence intensity of luciferases are extremely poor, it inevitably has required high sensitivity detectors, and has been considered to be inappropriate for single-cell imaging or exploration of organelles.

Meanwhile, as for fluorescent proteins, fluorescent proteins with multicolors have been studied well, and many facts regarding the coloring mechanisms thereof have been discovered. Therefore, based on these findings, many fluorescent proteins with diversified fluorescent characters have been developed. On the other hands, the palette of colors in luciferases is highly restricted. The advantages of diversification of bioluminescence colors include the follows: (i) simultaneous measurements of multiple cellular signals and (ii) In spite that advantages of red-shifted bioluminescence including its relieved tissue attenuation have been recited, a systematic study for diversifying the colors of bioluminescence has not been conducted.

Accordingly, it has been strongly desired to establish a new mutagenesis strategy exerting superluminescence and prolonged bioluminescence even in luciferases. Further, a well-designed study for red-shifted bioluminescence of luciferases is highly desired as well.

The present inventors have developed new bioluminescence imaging methods using Gaussia luciferase (GLuc) (Patent Literature 1), which is derived from Gaussia princeps as a marine animal, extremely small in the molecular weight, and exhibits higher bioluminescence intensities compared to other luciferase (Japanese Patent Application Nos. JP2008-116098, JP2009-025229 and JP2007-332253).

However, although its bioluminescence intensity is relatively high, the marine animal-derived luciferases including GLuc has not been popularly utilized as molecular probes, because the intensity of which were not as high as expected due to the intrinsic instability of bioluminescence and poor folding efficiency.

Although relatively many genetically mutated variants of Gaussia luciferase are known, none of the mutants have sufficient bioluminescence intensity and stability (Patent Literature 2 to 6). Although the anchor peptide (JP2009-025229) developed by the present inventors exerts considerably improved optical stability in the host probe, further enhancements in bioluminescence and stability have been highly desired.

### PRIOR ART DOCUMENTS

### Patent Literature

[Patent Literature 1] International Publication No. 1999-49019 (WO1999/049019)
[Patent Literature 2] Specification of U.S. Pat. No. 6,232,107
[Patent Literature 3] Specification of U.S. Pat. No. 6,436,682
[Patent Literature 4] Specification of U.S. Pat. No. 6,780,974
[Patent Literature 5] Specification of U.S. Pat. No. 7,045,599
[Patent Literature 6] Specification of U.S. Pat. No. 7,238,497
[Patent Literature 7] International Publication No. 2008-84869 (WO2008/084869)

### Non-Patent Literature

[Non-patent Literature 1] Awais, M.; Sato, M.; Lee, X. F.; Umezawa, Y.; Angew. Chem. Int. Ed. 2006, 45, 2707-2712.
[Non-patent Literature 2] Awais, M.; Sato, M.; Sasaki, K.; Umezawa, Y.; Anal. Chem. 2004, 76, 2181-2186.
[Non-patent Literature 3] Hoshino, H.; Nakajima, Y.; Ohmiya, Y.; Nature Methods 2007, 4, 637-639.
[Non-patent Literature 4] Paulmurugan, R.; Gambhir, S. S.; Anal. Chem. 2005, 77, 1295-1302.
[Non-patent Literature 5] Paulmurugan, R.; Gambhir, S. S.; Proc. Natl. Acad. Sci. U.S.A. 2006, 103, 15883-15888.
[Non-patent Literature 6] Kim, S. B.; Ozawa, T.; Watanabe, S.; Umezawa, Y.; Proc. Natl. Acad. Sci. U.S.A. 2004, 101, 11542-11547.
[Non-patent Literature 7) Kim, S. B.; Ozawa, T.; Umezawa, Y.; Anal. Chem. 2005, 77, 6588-6593.
[Non-patent Literature 8] Kim, S. B.; Awais, M.; Sato, M.; Umezawa, Y.; Tao, H.; Anal. Chem. 2007, 79, 1874-1880.
[Non-patent Literature 9] Kim, S. B.; Otani, Y.; Umezawa, Y.; Tao, H.; (2007) Anal. Chem., 79, 4820-4826.
[Non-patent Literature 10] Kim, S. B.; Umezawa, Y.; Kanno, K. A.; Tao, H.; ACS Chemical Biology 2008, 3, 359-372.
[Non-patent Literature 11] Kim, S. B.; Sato, M.; Tao, H.; Bioconjugate Chem. 2008, 19, 2480-2486.
[Non-patent Literature 12] Kim, S. B.; Sato, M.; Tao, H.; Anal. Chem. 2009, 81, 67-74.
[Non-patent Literature 13] Matthews, L.; Berry, A.; Tersigni, M.; D'Acquisto, F.; Ianaro, A.; Ray, D.; Endocrinology 2009, 150, 75-86.
[Non-patent Literature 14] Kim, S. B.; Sato, M.; Tao, H.; Anal. Chem. 2009, 81, 67-74.
[Non-patent Literature 15) Loening A. M.; Fenn T. D.; Gambhir S. S.; J. Mol. Biol. 2007, 374 (4), 1017-1028.
[Non-patent Literature 16] Cubitt A. B.; Woollenweber L. A.; Heim R.; Meth. Cell Biol. 1999, 58, 19-30.
[Non-patent Literature 17] Atsushi Miyawaki; Takeharu Nagai; Hideaki Mizuno; Curr. Opinion Chem. Biol. 2003, 7:557-562.
[Non-patent Literature 18] N. C. Shaner; G. H. Patterson; M. W. Davidson; J. Cell Sci., 2007 120 (24), 4247-4260.
[Non-patent Literature 19] Okita K.; Ichisaka T.; Yamanaka S.; Nature, 2007, 448 (7151) 313-317.
[Non-patent Literature 20] Papworth, C.; Bauer, J. C.; Braman, J; Wright, D. A.; Strategies, 1996, 9(3):3-4.

### SUMMARY OF INVENTION

### Technical Problem

An object of the present invention is to establish a method for enhancement of bioluminescence intensity, shifting the bioluminescence to a longer wavelength (red-shift) and improvement of bioluminescence stability of luciferase; and to provide an artificially-designed superluminescent variant of luciferase with prolonged bioluminescence by the method. Another object of the present invention is to establish a bioluminescence imaging method using the luciferase.

### Solution to Problem

In order to attain the above objects, the present inventors focused their attention on marine luciferases among various luciferases. On the whole, luciferases are extremely variable in the structures and share no considerable phylogenetical similarities. Therefore, it is difficult to determine generally acceptable rules among luciferases. However, upon spotlight on marine luciferases instead of whole luciferases, they share a common substrate and relatively high similarities in sequence. Owing to these advantages, the inventors speculated that, if the conventional knowledge regarding marine luciferases were combined, it may be possible to predict the structure of luciferase-substrate interactions with high probability, and to estimate the active sites of luciferases. On the basis of this speculation, the inventors attempted to enhance bioluminescence intensity, shift the bioluminescence to a longer wavelength (red-shift) and improve optical stability (i.e. prolonged bioluminescence of luciferase) by the modification of the putative active site and vicinity thereof. More specifically, the inventors paid attention to the DNA sequence of the smallest luciferase derived from Gaussia princeps (GLuc) among luciferases (GENE ACCESSION #: FJ010198). Firstly, the sequence was codon-optimized and modified to create many mutants by mutating the active site and adjacent amino acids thereof. Each variant was determined to be red-shifted luciferases with prolonged bioluminescence and enhanced intensity.

The observation revealed that conservative amino acid replacement of at least one of the amino acid residues at positions 89, 90, 95, 97, 100, 108, 112, 115, and 118 successfully improved activity of the luciferase, i.e., enhanced bioluminescence intensity of the luciferase with stabilized bioluminescence or red-shifted bioluminescence.

More specifically, significant improvements in luciferases' enzymatic properties such as the optical intensity, optical stability, and red-shifted feature of the bioluminescence are observed through replacing one or more amino acids carrying hydrophobic and nonpolar aromatic properties of the host luciferase at least one position selected from positions 89, 90, 97, 108, 112, 115, and 118 with other hydrophobic and nonpolar aromatic amino acid residue In particular, by individually replacing one or more hydrophobic nonpolar aliphatic amino acid residues at positions 90, 108, 112, 115, and 118 with other hydrophobic nonpolar aliphatic amino acid residues; or replacing aromatic amino residues at position 89 and/or 97 with other aromatic amino residue(s). Further, it was also confirmed that replacement of hydrophilic amino acid residues at position 95 and/or 100 forming a hydrogen bond with other hydrophilic amino acid residue exert a red-shifts of the bioluminescence.

Based on these findings, the present invention was completed.

Further, by introducing mutation into Metridia longa (MLuc) and Metridia pacifica 1 (MpLuc1), which are marine luciferases similar to GLuc, at the positions corresponding to the mutated sites of GLuc, an enhancement in luminescence intensity and a red-shift of the spectra similar to those exhibited by GLuc were observed. This proved that the above mutation sites are common performance-improving sites of marine luciferases.

Moreover, by using the new luciferase synthesized by the present invention as a reporter gene for reporter-gene assay or mammalian two-hybrid assay, or by using the luciferase as a dissected luciferase in bioluminescent probe method, strong and stable bioluminescence signal were determined in these assays with reduced assay times and improved S/N ratios. Among these, the usage of the luciferase variants of the present invention as a reporter-gene assay was found to be a revolutionary modification technique of hitherto known assays; therefore, as a separate invention, a patent application of this modification technology was filed with the Japan Patent Office on the same date as the application date of the present invention.

Specifically, the present invention comprises the following items.

### [Item 1]

A luciferase variant with improved optical property obtained by replacing at least one amino acid residue among the amino acid sequences of marine luciferases at positions corresponding to positions 89 to 118 in the amino acid sequence of Gaussia luciferase (GLuc), wherein an amino acid residue at a position corresponding to at least one selected from positions 89, 90, 95, 97, 100, 108, 112, 115, and 118 in the amino acid sequence of GLuc is replaced by way of conservative amino acid replacement.

### [Item 2]

The luciferase variant according to Item 1, wherein the conservative amino acid replacement includes at least one replacement of the following:
(i) replacement of a hydrophobic amino acid residue at a position corresponding to at least one selected from positions 89, 90, 97, 108, 112, 115, and 118 in the amino acid sequence of GLuc with another hydrophobic amino acid residue, and
(ii) replacement of a hydrophilic amino acid residue at a position corresponding to at least one selected from positions 95 and 100 in the amino acid sequence of GLuc that forms a hydrogen bond with another hydrophilic amino acid residue.

### [Item 3]

The luciferase variant according to Item 2, wherein the replacement of a hydrophobic amino acid residue with another hydrophobic amino acid residue includes at least one replacement of the following:
(i) replacement of a hydrophobic and nonpolar aliphatic amino acid residue at a position corresponding to at least one selected from positions 90, 108, 112, 115, and 118 in the amino acid sequence of GLuc with another hydrophobic and nonpolar aliphatic amino acid residue, and
(ii) replacement of a hydrophobic aromatic amino acid residue at a position corresponding to at least one selected from positions 89 and 97 in the amino acid sequence of GLuc with another hydrophobic aromatic amino acid residue.

### [Item 4]

The luciferase variant according to Item 3, wherein the replacement of a hydrophobic and nonpolar aliphatic amino acid residue with another hydrophobic and nonpolar aliphatic amino acid residue is replacement of isoleucine with an amino acid residue selected from leucine, tryptophan, and valine.

### [Item 5]

The luciferase variant according to Item 3, wherein the replacement of a hydrophobic aromatic amino residue with another hydrophobic aromatic amino residue is replacement of an amino acid residue at a position corresponding to at least one position selected from positions 89 and 97 in the amino acid sequence of GLuc with tryptophan (W).

### [Item 6]

The luciferase variant according to Item 2, wherein the replacement of a hydrophilic amino acid residue at a position corresponding to at least one selected from positions 95 and 100 in the amino acid sequence of GLuc that forms a hydrogen bond with another hydrophilic amino acid residue is at least one replacement selected from replacement of an amino acid residue corresponding to position 95 in the amino acid sequence of GLuc with glutamic acid (E), and replacement of an amino acid residue corresponding to position 100 in the amino acid sequence of GLuc with asparagine (N).

### [Item 7]

A luciferase variant obtained by way of conservative amino acid replacement that includes replacement of an amino acid residue corresponding to position 90 in the amino acid sequence of GLuc among the amino acid sequences of marine luciferase with leucine.

### [Item 8]

The luciferase variant according to Item 7, wherein the conservative amino acid replacement further includes at least one replacement selected from replacement of an amino acid residue corresponding to position 89 in the amino acid sequence of GLuc with tryptophan, and replacement of an amino acid residue corresponding to position 115 in the amino acid sequence of GLuc with leucine.

### [Item 9]

The luciferase variant according to Item 7 or 8, wherein the conservative amino acid replacement further includes replacement of an amino acid residue corresponding to position 95 in the amino acid sequence of GLuc with glutamic acid or glutamine, and replacement of an amino acid residue corresponding to position 97 in the amino acid sequence of GLuc with tryptophan.

### [Item 10]

The luciferase variant according to any one of Items 1 to 9, wherein the marine luciferase is one luciferase selected from Gaussia luciferase (GLuc) or a Copepod luciferase selected from MLuc, MpLuc1, and MpLuc2.

### [Item 11]

A DNA encoding the luciferase variant according to any one of Items 1 to 10.

### [Item 12]

An expression vector comprising the DNA according to Item 11.

### [Item 13]

A transformed cell in which the DNA according to Item 11 is introduced in a manner such that the DNA can be expressed.

### [Item 14]

A method for improving optical property of marine luciferase, comprising replacing at least one amino acid residue among the amino acid sequence of a marine luciferase at positions corresponding to position 89 to 118 in the amino acid sequence of Gaussia luciferase (GLuc), wherein an amino acid residue at a position corresponding to at least one selected from positions 89, 90, 95, 97, 100, 108, 112, 115, and 118 in the amino acid sequence of GLuc is replaced by way of conservative amino acid replacement.

### [Item 15]

A bioluminescent probe with improved optical property that adopt a working mechanism of resurrecting luminescent function by reconstituting dissected luciferase fragments, the probe using the luciferase variant according to any one of Items 1 to 10 as the luciferase.

[Item 16] A DNA encoding a bioluminescent probe according to Item 15.

### Advantageous Effects of Invention

The present invention successfully improved the activity of luminescence enzyme, i.e., enhanced luminescence intensity in a longer wavelength region due to enhanced luminance of luciferase or a shift to longer wavelength, and also attained an enhanced stability, only by replacing at least one amino acid residue in the luminescent enzyme active region and in the vicinity thereof conserved in a wide range of general marine luciferases including GLuc, with a hydrophobic amino acid residue, an aromatic amino acid residue and/or an amino acid residue that forms a hydrogen bond.

By being used as an element of a luciferase for hitherto well-known reporter gene assay, yeast two-hybrid assay, mammalian two-hybrid assay, protein splicing assay, protein complementation assay, circular permutation assay, bioluminescence resonance energy transfer (BRET) assay (Non-patent Literature. 3 to 12) or the like, the superluminescent marine luciferase of the present invention can create a luminescent probe with high luminance and stability, thereby significantly improving measurement performance in these assays. It should be noted that the disclosures of the prior art documents and specifications of patent applications referred to in the present invention are included in the disclosure of the specification of the present invention.

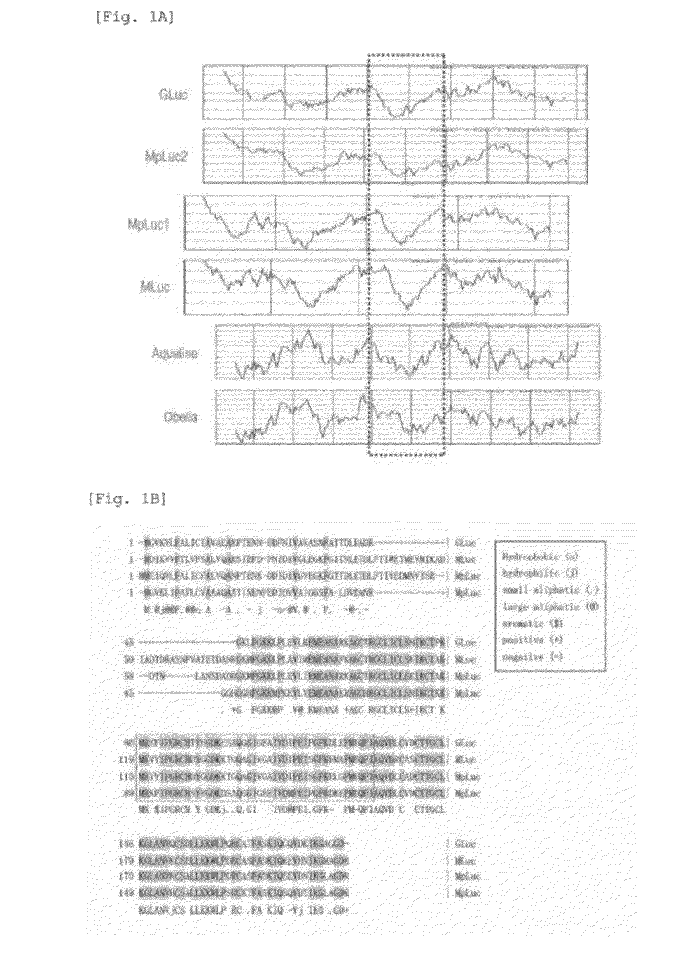

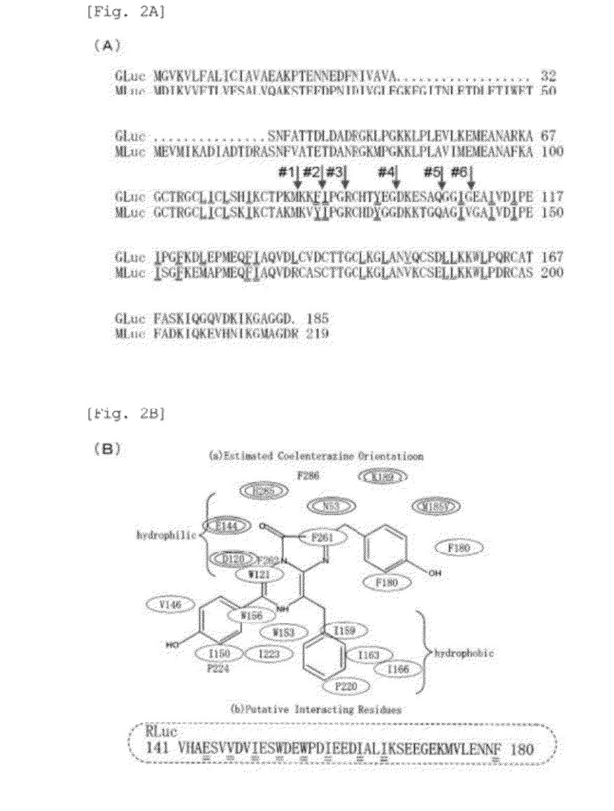

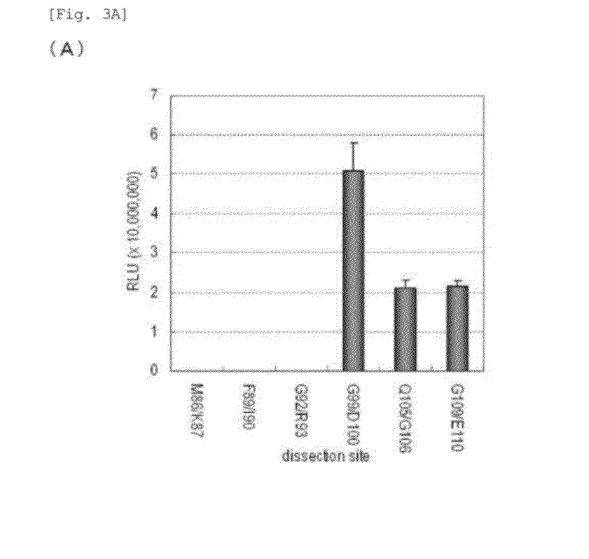

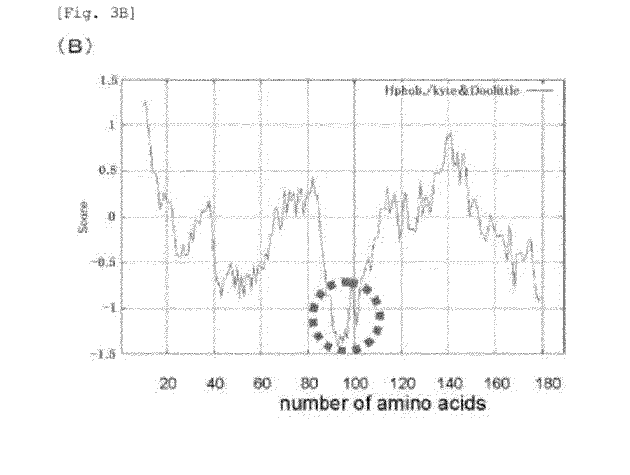

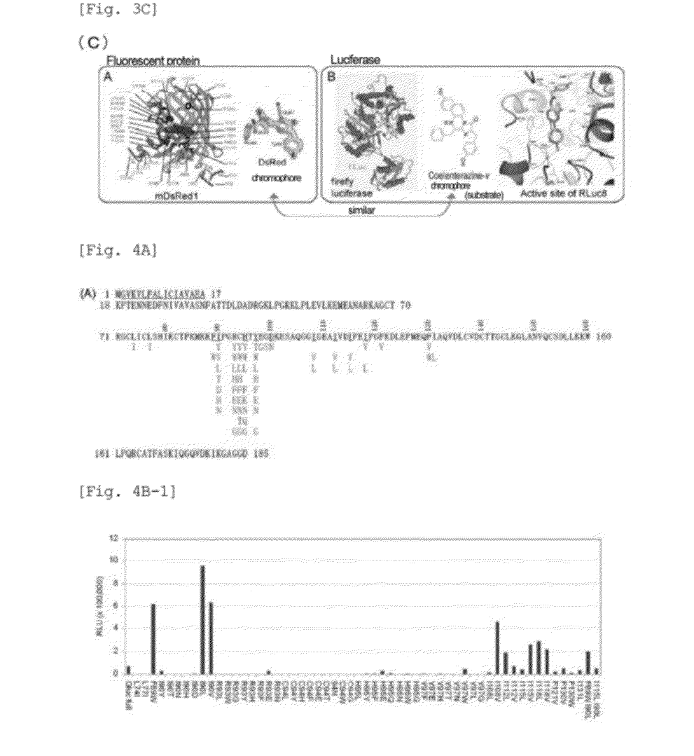

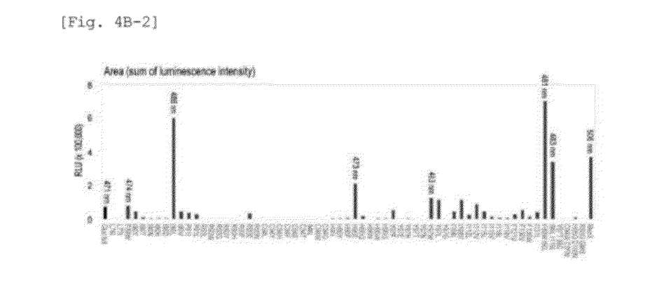

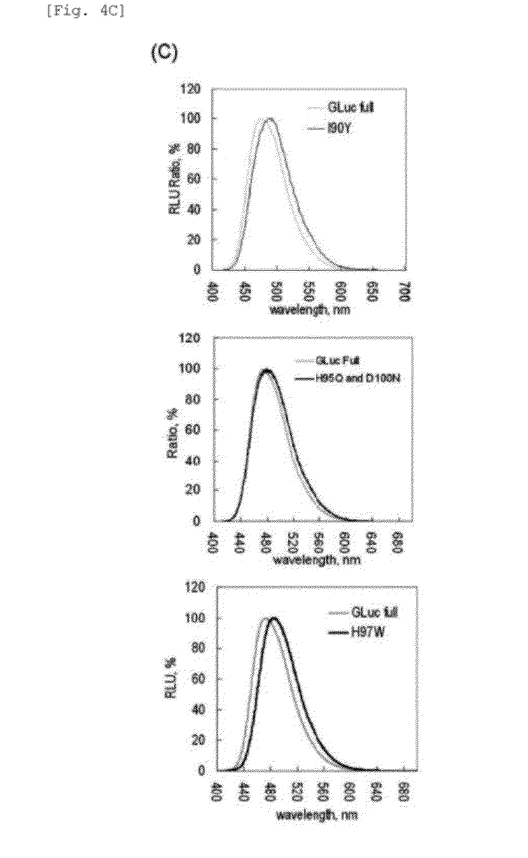

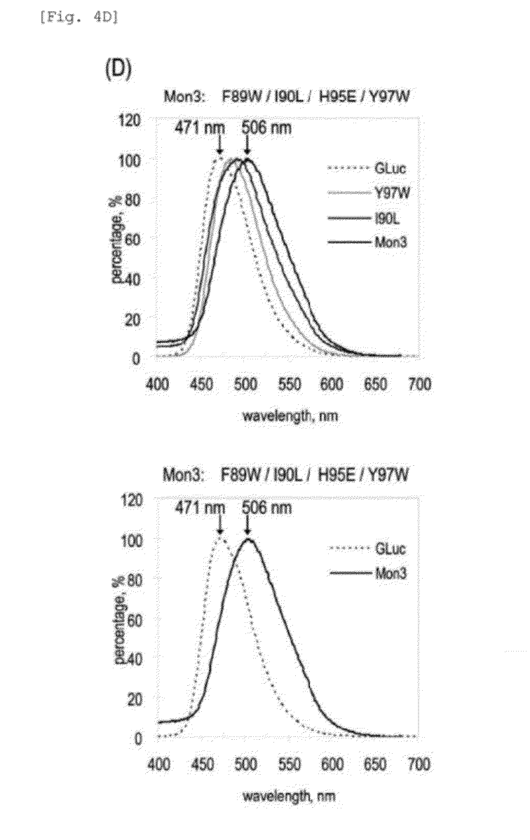

### BRIEF DESCRIPTION OF DRAWINGS

[FIG. 1A] A graph showing hydrophobicity distribution in each amino acid sequence of various marine luciferases.

[FIG. 1B] A comparative chart of amino acid sequences of various marine luciferases. The dotted box denotes a putative enzyme active site.

[FIG. 2A] A chart showing the entire amino acid sequence of Gaussia luciferase (GLuc). The sequence of GLuc was compared with the sequence of a luciferase derived from Metridia longa (MLuc). The arrow indicates the enzyme dissection sites in the present study.

[FIG. 2B] A chart showing a known crystallographic structure showing the active site of Renilla luciferase. The chemical substance in the middle shows a molecular structure of the specific substrate (coelenterazine). The respective positions of amino acids show that the active site generally resides between the group of hydrophobic amino acids (single lined oval circle) and the group of hydrophilic amino acids (double lined oval circle).

[FIG. 3A] A graph showing recovery level of bioluminescence intensity at each dissection site of GLuc.

[FIG. 3B] A chart showing distribution of hydrophobic amino acids of GLuc. A hydrophilic region covers the vicinity of amino acid numbers 90 to 100.

[FIG. 3C] A drawing for comparing chromophores of fluorescent protein and luminescent protein. Section A shows the crystal structure of fluorescent protein and the chemical structure of the chromophore. Section B shows, from left to right, the crystal structure of firefly luciferase, the chemical structure of coelenterazine, and the crystal structure of Renilla luciferase, respectively.

[FIG. 4A] A map showing mutation sites in the amino acid sequence of GLuc.

[FIG. 4B-1] A graph (1) showing a comparison among different GLuc variants with respect to bioluminescence intensities. The graph compares peak values of luminescence spectra measured by a spectrophotometer, showing relative bioluminescence intensities upon comparison with the luminescence intensity of native GLuc (the leftmost value). Here, the cell dissolution time was set to 5 minutes, the time of which is not enough to dissolve organelles.

[FIG. 4B-2] A graph (2) showing a comparison among different GLuc variants in terms of bioluminescence intensity. The graph compares areas of luminescence spectrum measured by a luminescence plate reader, showing relative bioluminescence intensities based on the original luminescence intensity (the leftmost value). Here, the cell dissolution time was set to 20 minutes, thereby dissolving organelle to obtain uniform lysis solutions. The graph shows maximum values of luminescence wavelengths for the variants with high luminances.

[FIG. 4C] A graph showing a redshift of bioluminescence spectrum of GLuc variants. The upper graph shows mutation of I90 into Tyr(Y), the middle graph shows mutation of H95 and D100 into Q and N, respectively, and the lower graph shows mutation of H97 into W.

[FIG. 4D] A graph showing redshifts of bioluminescence spectra of GLuc variants. The graph shows each luminescence spectrum of Y97W, I90L, and F89W/I90L/H95E/Y97W (Mon3), compared with a conventional GLuc.

[FIG. 4E] A graph showing relative long wavelength bioluminescence intensities measured by a 610 nm long-pass filter. The graph shows respective wavelengths of luminescence variants compared with a known GLuc (white bar). The graph reveals that the variants show strong bioluminescences at long wavelengths.

[FIG. 5] Photos comparing luminescence intensities of GLuc variants with significantly strong bioluminescences.

(A) A digital photo showing the positions of luminescent tubes. The photo shows lysis solutions expressing, from left to right, firefly luciferase (FLuc), known Gaussia luciferase (GLuc), and I90L variant of GLuc.

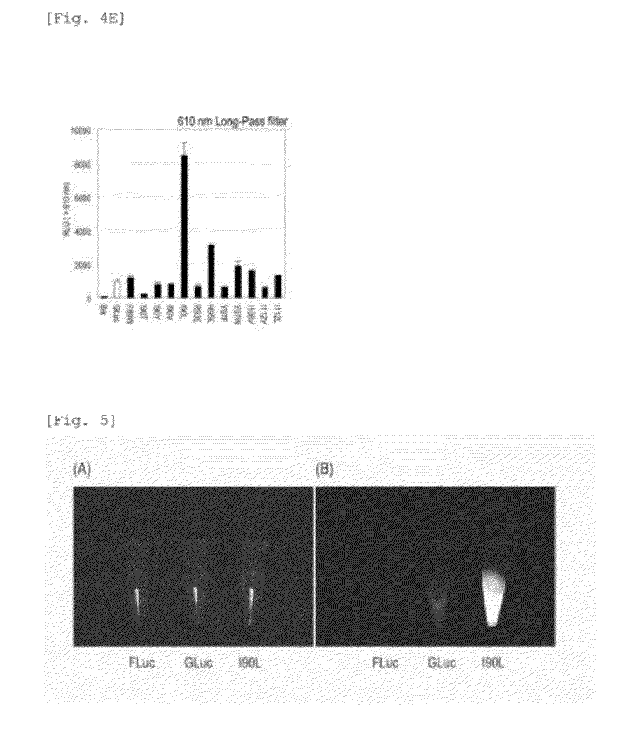

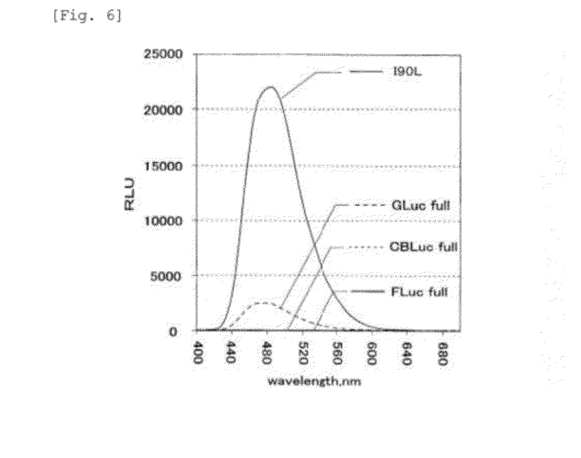

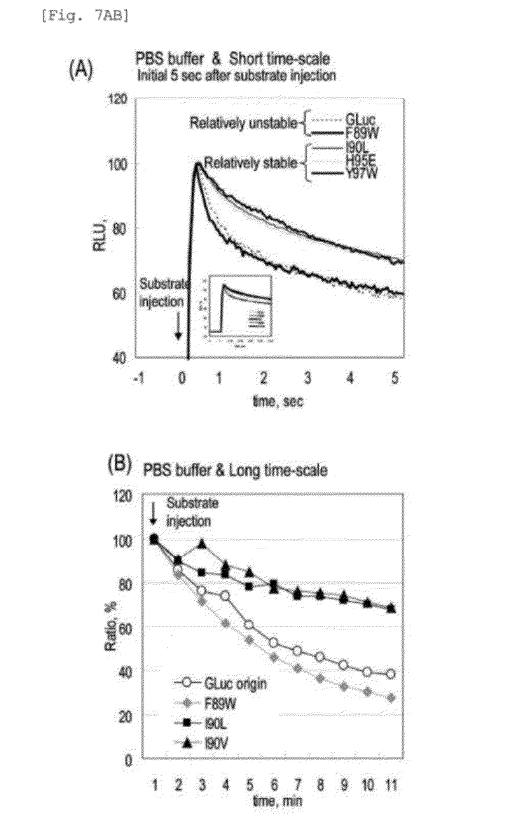

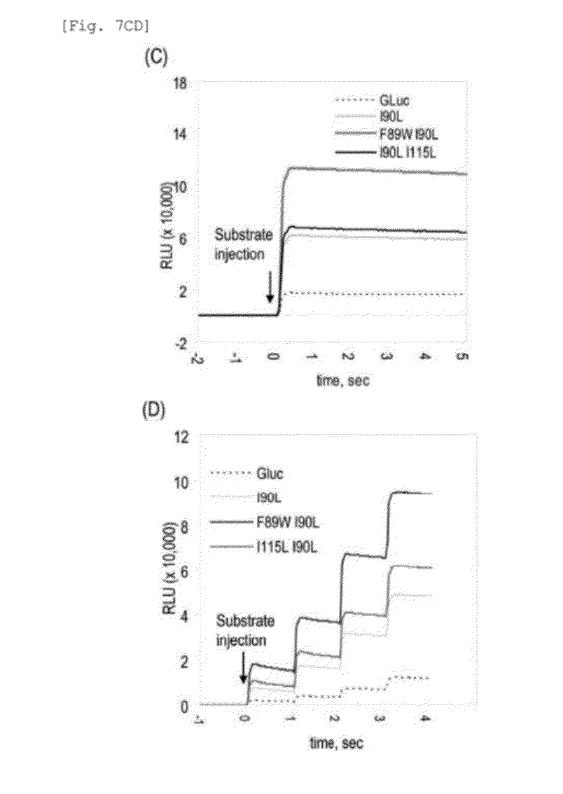

(B) A photo comparing luminescence levels of respective tubes. The photo reveals that the variant I90L of GLuc on the far right has the strongest bioluminescence.

[FIG. 6] A graph comparing bioluminescence spectrums of typical luciferases. The graph reveals that the bioluminescence intensity of the GLuc variant (solid line) is far stronger than those of known GLuc (dotted line), FLuc, and click beetle luciferase (CBLuc).

[FIG. 7AB] Graphs showing changes in bioluminescence intensity with time.

(A) The graph shows a result of a short-time (5 seconds) observation of changes in bioluminescence with time after introducing the substrate (dissolved in PBS buffer) into each variant. The graph shows that I90L, H95E, and Y97W exhibit relatively stable bioluminescences.

(B) The graph shows a result of a relatively long-time (10 minutes) observation of changes in bioluminescence with time after introducing the substrate (dissolved in PBS buffer) into each variant. The graph reveals that the luminescence intensities (white circle) of the original GLuc significantly decreased, whereas the luminescence intensities of I90L (black square) and I90V (gray triangle) are relatively stable.

[FIG. 7CD] Graphs showing changes in bioluminescence intensity with time.

(C) The graph shows a result of a short-time (5 seconds) observation of changes in bioluminescence with time after introducing the substrate (dissolved in a modified Matthew's buffer into each variant.

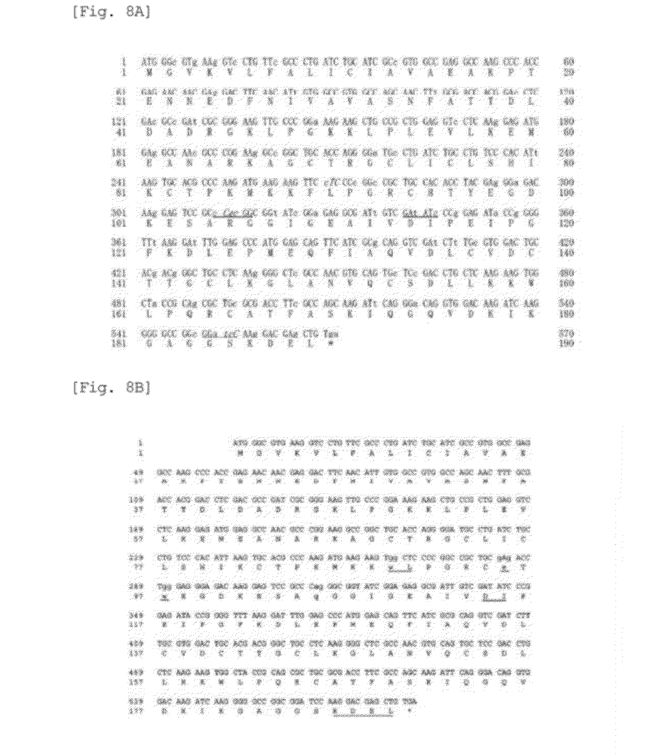

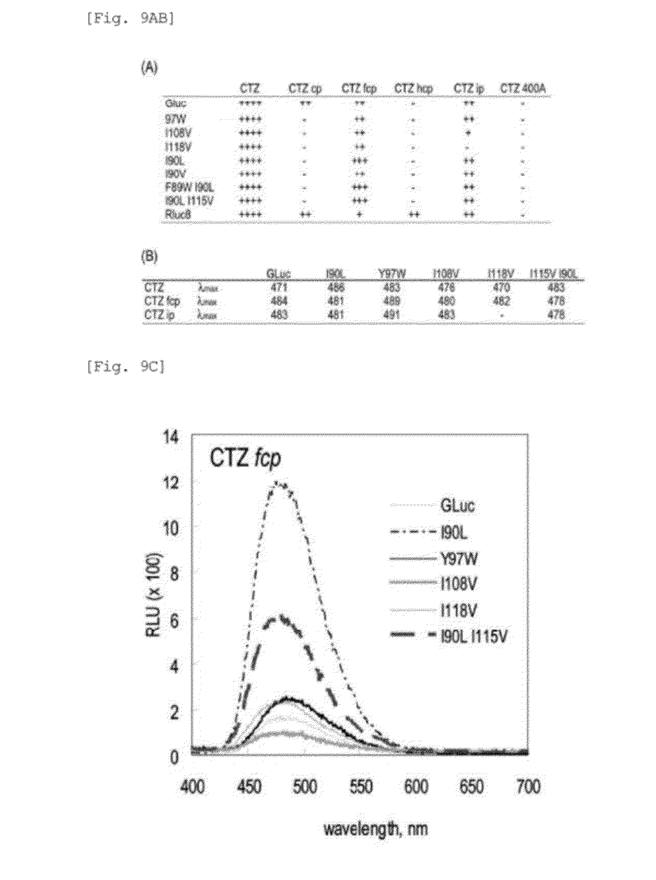

(D) The graph shows a result of observation of changes in bioluminescence with time that was conducted per second for each stage after introducing the substrate (dissolved in a modified Matthew's buffer) into each variant stepwise. The graph reveals that all variants are very stable, showing strong bioluminescences.

[FIG. 8A] A drawing showing a base sequence modification example (1) of a GLuc variant suitable for expression in mammals including human. FIG. 8(1) shows a base sequence of a variant I90L of GLuc consisting of codons suitable for mammals including human, to which a restriction enzyme was introduced. The underline shows the restriction enzyme site, and the italicized letters indicate the mutation sites.

[FIG. 8B] A drawing showing a base sequence modification example (2) of a GLuc variant suitable for expression in mammals including human. FIG. 8(2) shows a base sequence of GLuc enzyme variant (Mon3 with four mutations including I90L) consisting of codons suitable for mammals including human, to which an EcoRV restriction enzyme was introduced. The underline shows the restriction enzyme site, and the italicized letters indicate the mutation sites.

[FIG. 9AB] Tables showing substrate selectivities of the GLuc variants of the present invention.

(A) The table shows comparison between the respective variants in terms of substrate selectivity using their luminescence intensities as index. The table shows levels of relative luminescence intensities based on the standard substrate (coelenterazine; CTZ) shown as “++++.”

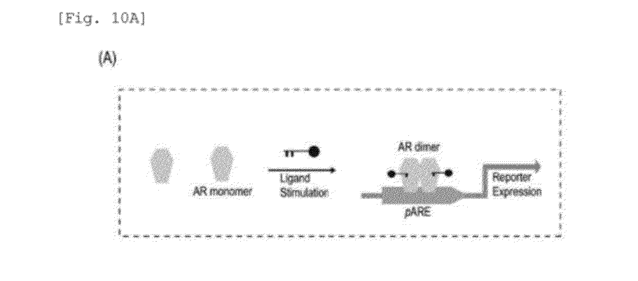

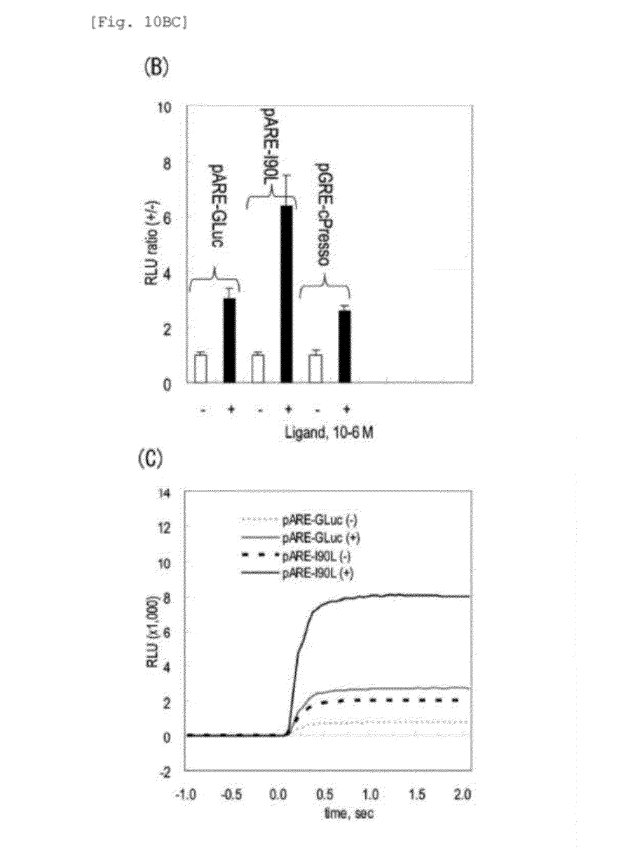

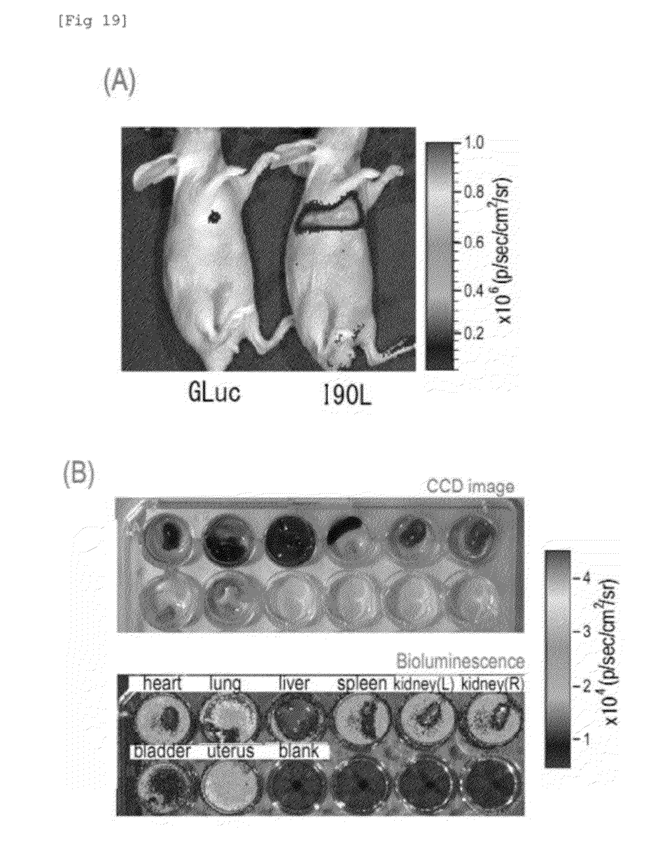

(B) The table shows the maximum luminescence wavelength (λmax) of luminescence spectrum for each synthetic coelenterazine. The table reveals that each variant has a different luminescence color depending on the type of synthetic coelenterazine.

[FIG. 90] Graph showing substrate selectivity spectrums of the GLuc variants of the present invention. The graph shows, as an example, bioluminescence spectrums of the variants in the presence of coelenterazine fcp.

[FIG. 10A] A drawing showing reporter gene assay based on the GLuc variants of the present invention.

(A) The drawing shows the principle of the assay. The androgen receptor is activated by ligand stimulation (androgen), duplicated, and then bound to an androgen responsive element (ARE), thereby expressing the reporter gene (GLuc variant).

[FIG. 10BC] The graphs showing a reporter gene assay based on the GLuc variants of the present invention.

(B) The graph shows bioluminescence intensities in the presence and absence of androgen. The graph reveals that the GLuc variant (I90L) shows an excellent signal-to-noise ratio (S/N ratio) compared with a known GLuc.

(C) The graph shows changes in bioluminescence with time of the expressed reporter protein. The graph reveals that both the intensity and stability of the luminance were superior when I90L was incorporated.

[FIG. 10DE] Graphs showing a reporter gene assay based on the GLuc variants of the present invention.

(D) The graph shows a comparison in terms of luminescence intensity in the reporter gene assay between the case of using a known Gaussia luciferase (GLuc) and the case of using GLuc variant (I90L) of the present invention. The graph reveals that the area of the luminescence intensity was greater in case of using I90L.

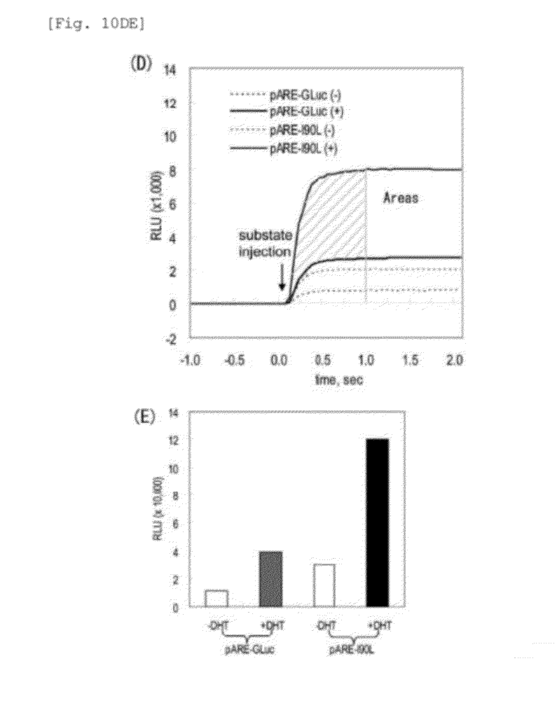

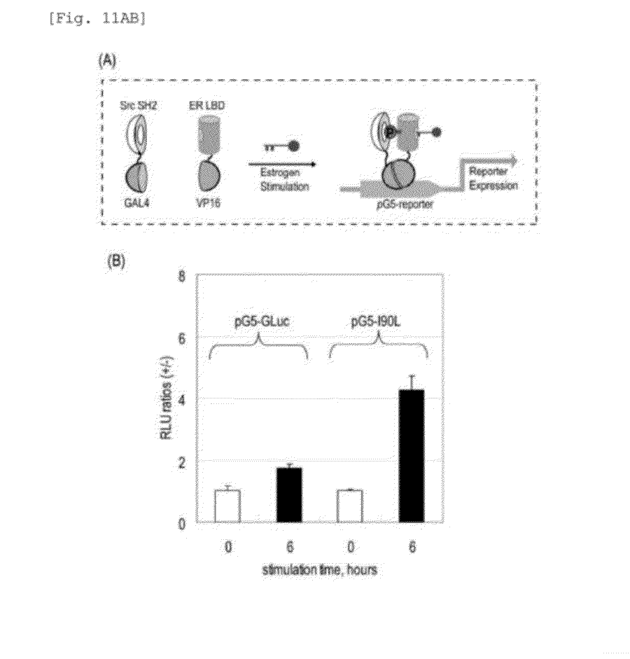

(E) The graph shows comparison of luminescence areas obtained in (D).

[FIG. 11AB] A graph showing a mammalian two-hybrid assay based on the GLuc variants of the present invention.

(A) The drawing shows the principle of two-hybrid assay. Src SH2 domain and ER LBD were used as the model proteins. The SH2 domain was ligated to Gal4, and the ER LBD was ligated to VP16. They were bound to each other by stimulation of an estrogen. In response to this, the GAL responsive element of pG5 induced expression of the reporter protein (GLuc variant).

(B) The graph shows changes in bioluminescence intensity depending on the estrogen stimulation time. A sufficient bioluminescence was observed by 6-hour estrogen stimulation when using GLuc variant of the present invention.

[FIG. 11CD] A graph showing mammalian two-hybrid assay based on the GLuc variants of the present invention.

(C) The graph shows changes in bioluminescence intensity with time when using the original GLuc (pG5-GLuc).

(D) The graph shows changes in bioluminescence intensity with time when using the GLuc variant of the present invention. The graph reveals that the absolute luminescence of the Gluc variant was higher than GLuc, and that the bioluminescence thereof was efficiently enhanced even by a short-time estrogene stimulation.

[FIG. 11E] A graph showing a mammalian two-hybrid assay based on the GLuc variants of the present invention.

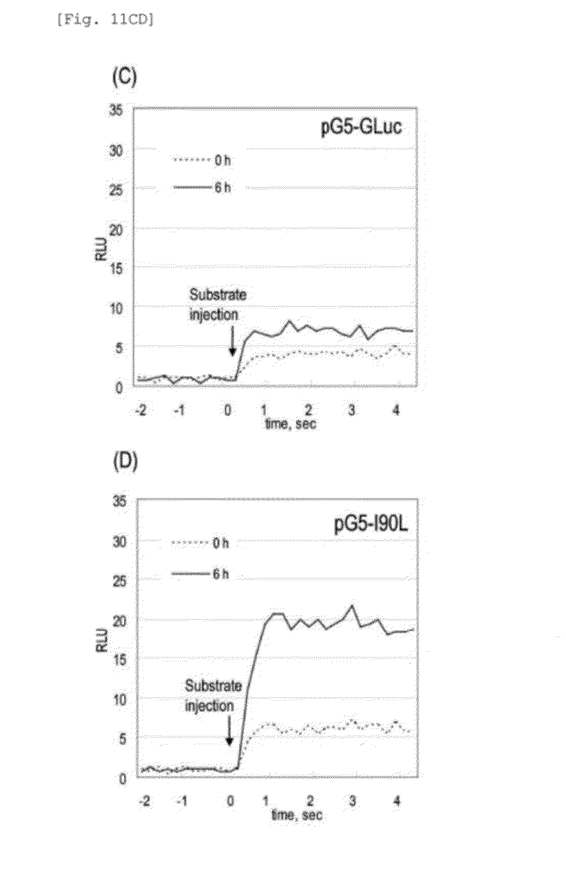

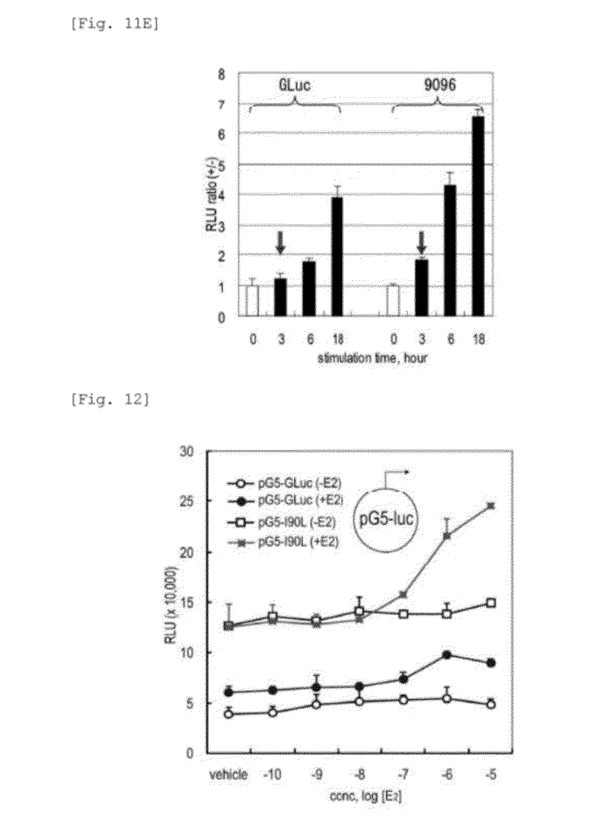

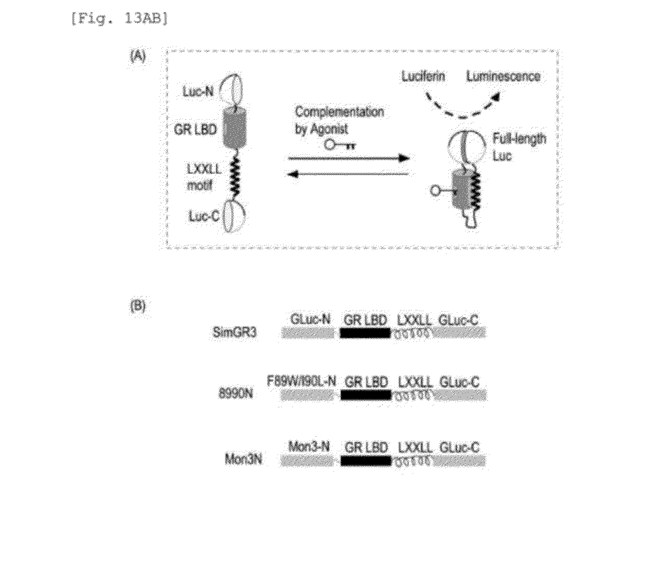

(E) The graph shows bioluminescence intensities (measured by a luminometer) after (after 0 hour, 3 hours, 6 hours, and 18 hours) estrogene stimulation when incorporating GLuc and GLuc variant (9096) in a pG5 vector.

[FIG. 12] A graph showing the results of a mammalian two-hybrid assay using the GLuc variant of the present invention. The graph shows that the bioluminescence intensity of the GLuc variant of the present invention was significantly enhanced depending on the concentration of estrogene.

[FIG. 13AB] A drawing showing a single-molecule-format bioluminescent probe using the GLuc variant of the present invention.

(A) The GLuc variant was dissected into two fragments, and a stress hormone receptor (glucocorticoid receptor) and a LXXLL motif were inserted between the fragments. A GLuc-based probe (“SimGR3”) was used as a control. In the figure, “8990N” denotes a GLuc variant having mutations at F89W/I90L, and “Mon3N” denotes a GLuc variant having mutations at F89W/I90L/H95E/Y97W.

(B) The drawing shows a molecular structure diagram of single-molecule-format bioluminescent probes containing GLuc variants of the present invention.

[FIG. 13CD] Graphs showing data regarding single-molecule-format bioluminescent probes containing GLuc variants of the present invention.

(C) The graph shows a response to stress hormone for each single-molecule-format bioluminescent probe containing the GLuc variant of the present invention. “E990N” exhibited a relatively strong bioluminescence in response to stress hormone.

(D) The graph shows changes in bioluminescence intensity with time of “8990N.” “8990N” exhibited strong bioluminescence in the presence of stress hormone.

[FIg. 13E] A graph showing data regarding single-molecule-format bioluminescent probes containing GLuc variants of the present invention.

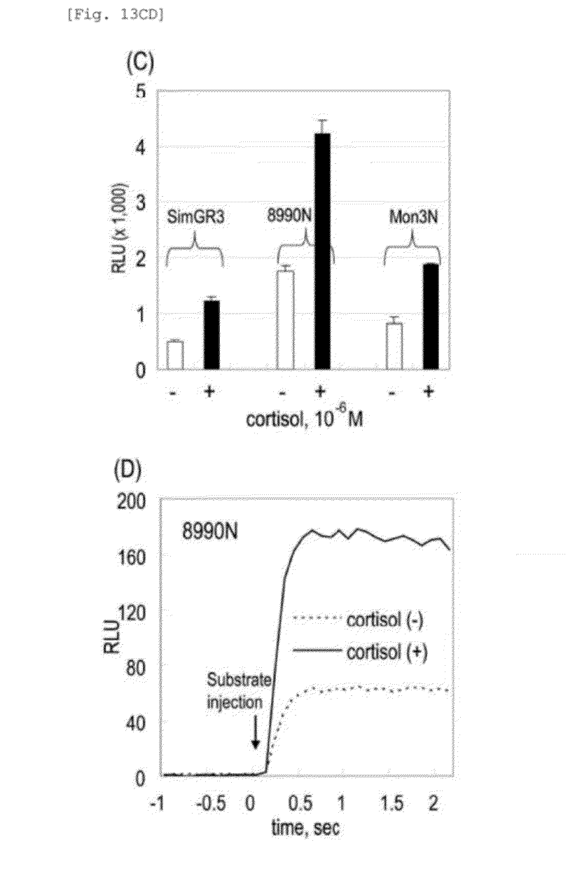

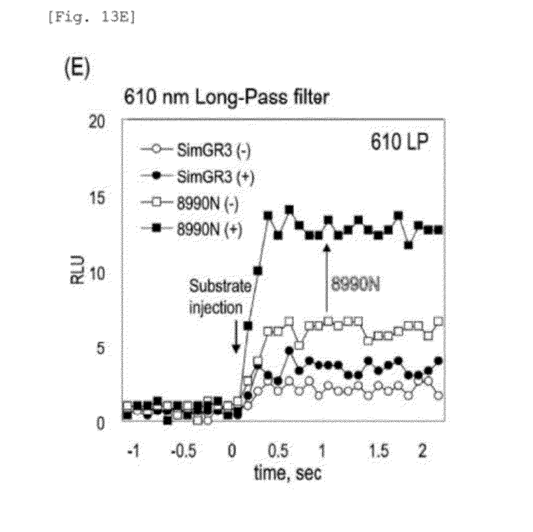

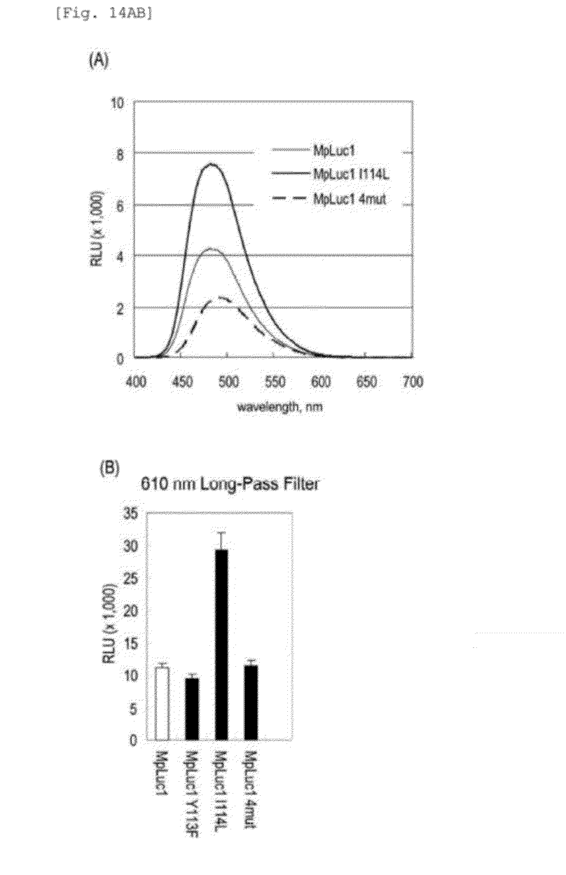

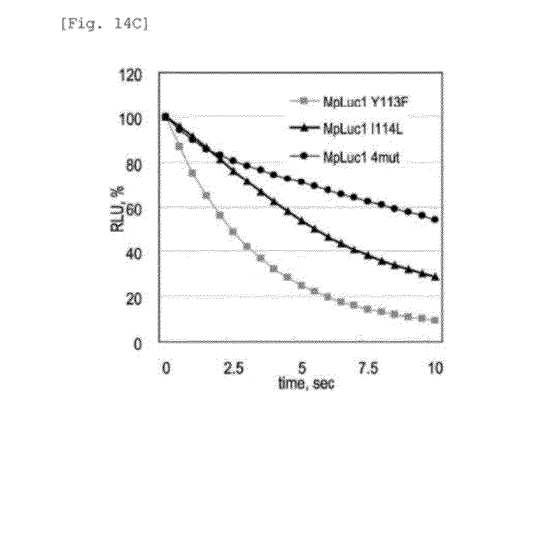

(E) The graph shows measurement of relative stress hormone sensitivities of SimGR3 and 8990N measured by a luminescence plate reader equipped with a 610-nm long-pass filter. The bioluminescence of “8990N” was greater, showing a higher sensitivity to stress hormone stimulation.

[FIG. 14AB] Graphs showing luminescence characteristics of MpLuc1 (Metridia pacifica-derived luciferase) variant.

(A) The graph shows luminescence spectrums of MpLuc1 itself, a I114L variant of MpLuc1 (I114L), and a Y113W/I114L/H119E/Y121W variant of MpLuc1 (MpLuc4).

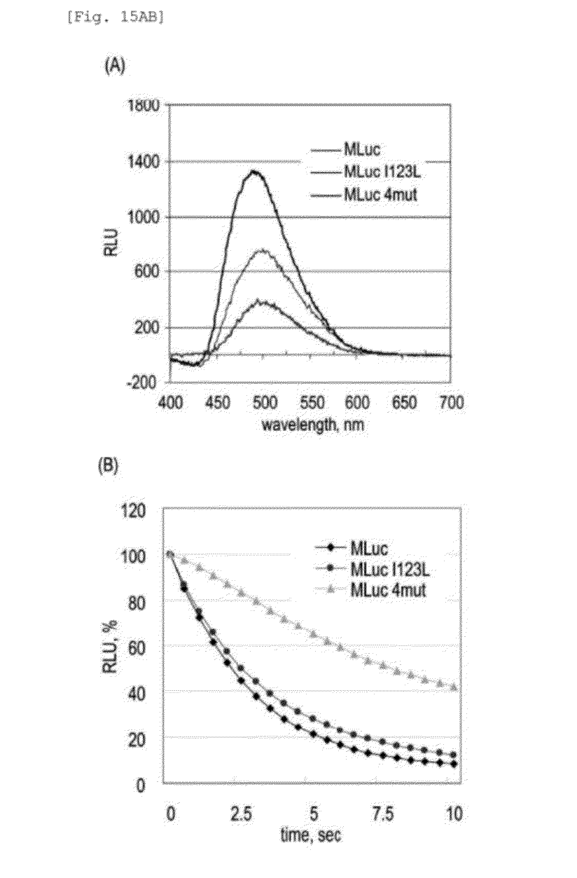

(B) The graph shows measurement of bioluminescence intensity of MpLuc1 variant with respect to a long wavelength (610 nm or more) using a 610-nm long-pass filter.

[FIG. 14C] The graph shows measurements of luminescence stability of the MpLuc1 variants. The graph reveals that both I114L and MpLuc4 variants showed more stable bioluminescences compared with the known MpLuc1.

[FIG. 15AB] Graphs showing luminescence characteristics of MLuc variants (Metridia longa-derived luciferase).

(A) The graph shows luminescence spectrums of MLuc itself, a I123L variant of MLuc (I123L), and a Y122W/I123L/H128E/Y130W variant of MLuc (MLuc4).

(B) The graph shows measurement of luminescence stabilities of MLuc variants. The graph reveals that the Mluc4 variant showed more stable bioluminescence compared with the known MLuc itself.

[FIG. 15CD] Graphs showing luminescence characteristics of MLuc variant.

(C) The graph shows measurement of bioluminescence intensities of MLuc variants with respect to a long wavelength (610 nm or more) using a 610-nm long-pass filter.

(D) The graph shows changes in luminescence intensity with time around substrate introduction.

[FIG. 16] (A) A photograph for observation of the animal tissue permeability of bioluminescence by a GLuc variant (8990) of the present invention carrying mutations at F89W/I90L. A conventional GLuc and 8990 were expressed at the subcutaneous layers of the left back and the right back, respectively, of a living mouse. The observation revealed that the bioluminescence from 8990 (right) was stronger than that from GLuc (left).

(B) A photograph exhibiting bioluminescence intensities from a two-hybrid assay system in the tissues of living animals when the GLuc variant is inserted into the system. Transformed COS-7 cells, that were cotransfected by pG5-GLuc or pG5-90L in addition to the common plasmids, pACT-SH2 and pBIND-ER LBD, was implanted into the subcutaneous layers of the left and right backs of a mouse. The measurement was performed in the presence or absence of estrogene. A stronger bioluminescence was observed in the layer where pG5-90L was introduced (right: expression of the I90L variant of GLuc).

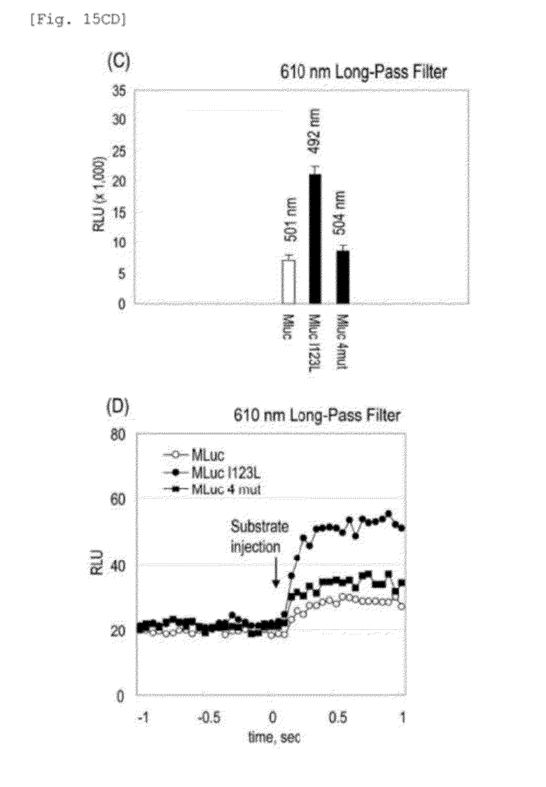

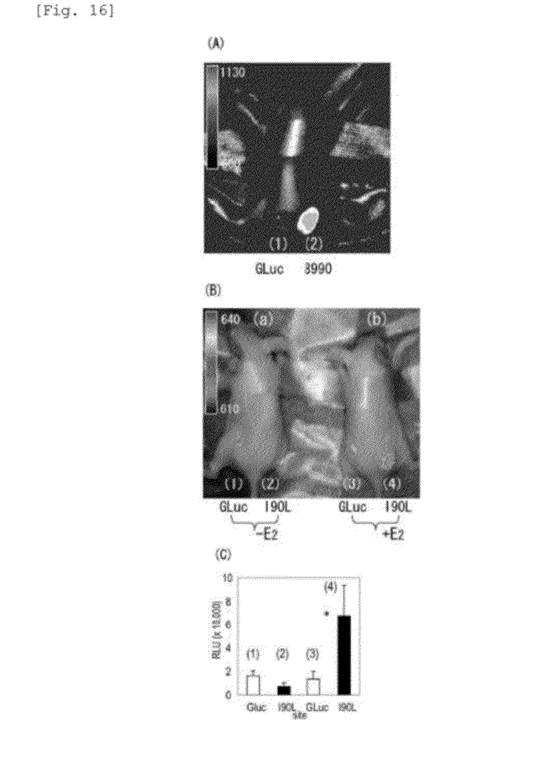

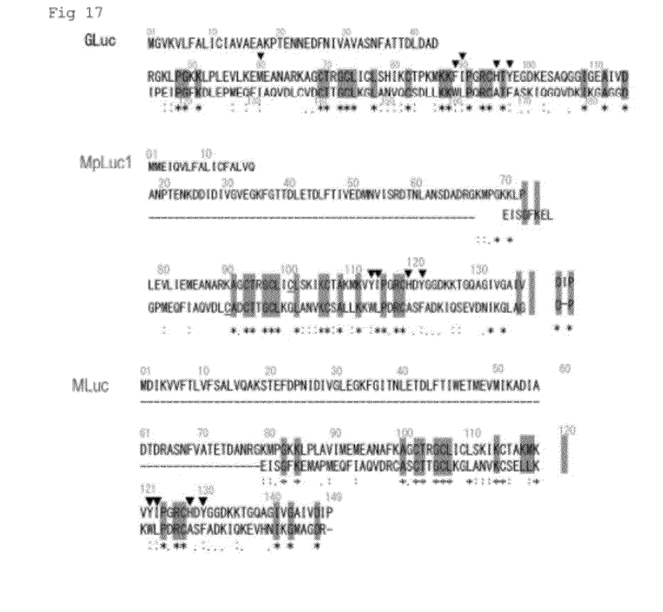

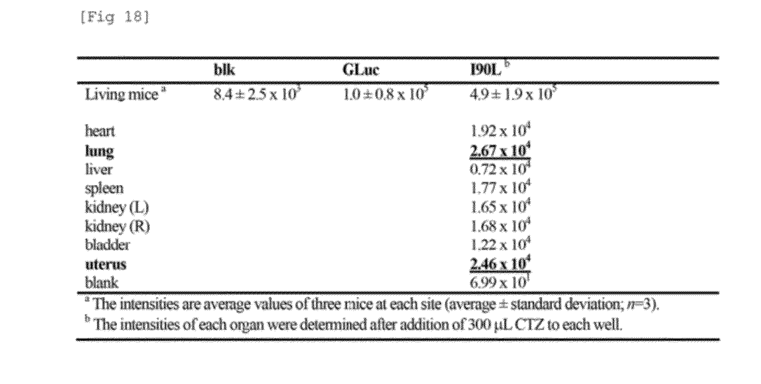

(C) A graph comparing relative luminescence intensities at the respective transplantation portions of the mouse in the above experiment (B). The values correspond to the sites (1), (2), (3), and (4), respectively, from left to right.

[FIG. 17] Single-Sequence Alignment (SSA) of GLuc, MpLuc1 and MLuc. The arrowheads clearly suggest putative mutation sites to increase consensus amino acids.

[FIG. 18] Table showing bioluminescence intensities in the cell-implanted sites of living mice and organs. The intensities demonstrate metastasis of murine B16 melanoma cells to organs in living mice. The unit is photons/sec/cm²/sr. The boldfaces highlight significant bioluminescence intensities.

[FIG. 19] (A) Bioluminescent imaging of metastasis of murine B16 melanoma cells expressing GLuc and I90L in living mice.

(B) Detailed histological analysis of metastasis of murine B16 melanoma cells in mice. After intravenous (iv) injection of B16 melanoma to a mouse via tail vein, the organs were isolated from the mouse and imaged in the presence of 300 μL CTZ.

### DESCRIPTION OF EMBODIMENTS

### 1. Marine Luciferase

In the present invention, “marine luciferase” mainly designates a luciferase produced by a “luminescent marine animal.” Examples of luminescent marine animals include genus Gaussia such as Gaussia princeps; genus Renilla such as Renilla reniformis; genus Cypridina such as Cypridina noctiluca; genus Vargula such as Vargula hilgendorfii; and genus Metridia such as Metridia longa and Metridia pacifica. Particularly preferable examples of “marine luciferase” include Gaussia luciferase (GLuc) and Metridia luciferase (such as MLuc derived from Metridia longa, or MpLuc1 and MpLuc2 which are luminescent enzyme groups derived from Metridia pacifica). Other known examples of marine luciferase include Obelin, a kind of luminescent plankton, aqualine Pleuromanma, and Oplophorus. These luminescent enzymes are very similar in terms of distribution of hydrophilic and hydrophobic amino acids in the entire enzyme; moreover, they have high similarity in amino acic sequence in the putative enzymatic activity region (FIG. 1). Therefore, the luciferase group is simply called “marine luciferase,” or specifically called “Gaussia-like luciferase.” The luciferase group also includes luciferase variants having a homology of 80% or more, preferably 90% or more, more preferably 95% or more in the amino acid sequences and/or the base sequences with those of GLuc (Gene Bank Assession Number: AY015993), MLuc (Gene Bank Assession Number: AY364164), MpLuc1 (Gene Bank Assession Number: AB195233), or MpLuc2 (Gene Bank Assession Number: AB195234). These Gaussia-like luciferases can be easily obtained by a homology search for the base sequence of the aforementioned GLuc, MLuc, MpLuc1, or MpLuc2 in the PSI-BLAST database or a similar database. It is also possible to establish those luciferases from natural Gaussia-like marine animal genes using a probe or primer based on those base sequences.

Although MLuc, MpLuc1, and MpLuc2 as Gaussia-like luciferases are slightly different from GLuc in their greater molecular weight than GLuc, they have very similar sequences, as explained above. Further, they are almost identical to GLuc in terms of enzymatic characteristics such as the substrate or luminescence activity. Therefore, the following description mainly focuses Gaussia luciferase (GLuc) as the representative Gaussia-like luciferase. Any finding regarding GLuc may also be applied to other Gaussia-like luciferases.

Hereinafter, the mutation site is indicated based on the amino acid sequence of GLuc, unless otherwise specified. For example, “amino acid 90 of GLuc” corresponds to the amino acid 123 of MLuc. It also corresponds to the amino acid 114 of MpLuc1 and 93 of MpLuc2. More specifically, for example, the amino acid residues corresponding to positions 89, 90, 95, 97, 100, 108, 112, 115 and 118 in the amino acid sequence of GLuc respectively correspond to the following amino acid residues.
Amino acid residues at positions 122, 123, 128, 130, 133, 141, 145, 148 and 151 of MLuc;
Amino acid residues at positions 113, 114, 119, 121, 124, 132, 136, 139 and 142 of MpLuc1; and
Amino acid residues at positions 92, 93, 98, 100, 103, 111, 115, 118 and 121 of MpLuc2.

Gaussia luciferase (GLuc) has the following characteristics.
(a) GLuc is the smallest luciferase ever discovered. Therefore, the use of GLuc for molecular imaging is much less burdensome for the host cell or host protein.
(b) GLuc has significant resistance to pH, surfactant, and chemical denaturant.
(c) GLuc has the highest luminance among luciferases.
(d) GLuc has a quick turnover cycle of enzyme, seven times or more than that of known firefly luciferase.

On the other hand, the bioluminescence of GLuc considerably depends on the reaction conditions, and significant decreases in intensity can easily occur, thereby degrading its stability. Therefore, GLuc was considered inappropriate as a reporter (index) for luminescence analysis that strictly requires stability.

### 2. Modification of Marine Luciferase of the Present Invention to Improve Luminescent Function

### (2-1) Enhancement in Optical Properties of Marine Luciferase

In the present invention, “enhancement in optical property of marine luciferase” mainly means enhancement in bioluminescence intensity and improvement in stability of bioluminescence intensity with time, which are important in the observation of reporter gene expression. However, it also encompasses a shift of the luminescence wavelength to a longer wavelength side (red-shift of spectra). This is an important nature to expand the utilization of the reporter gene, because the red-shift enhances transmittance through cells or skin.

The two-dimensional distribution of bioluminescence intensity can be obtained by measuring bioluminescence intensity in a specific wavelength range after addition of the substrate using a known luminescence spectrophotometer. By performing such measurement with time, bioluminescence stability with time may also be measured. A luminescence plate reader, which has superior sample processing property, may also be used to obtain more accurate data. Here, the areas of respective luminescence spectrums are measured after sufficient cell lysis time (about 20 minutes). The red-shift may be measured by a wavelength scanning method, or using a long wavelength filter.

### (2-2) Modification Method

The modification of the amino acid sequence of the marine luciferase of the present invention may be performed by causing a chemical change directly on the desired amino acid residue or residues of the amino acid sequence; however, the modification is generally performed by point mutation of the base corresponding to the target amino acid among the base sequence encoding the enzyme. The mutation of the base may be performed by a well-known method, such as site-directed point mutation. The following description discusses the case of introducing point mutation according to the “Quick-Change method” (Non-patent Literature 20); however, the present invention is not limited to this method.

More specifically, the introduction of mutation is conducted by (i) performing PCR reaction in the co-existence of a pair of sense primer and antisense primer of about 30 bases prepared to have a mutation point with a E-coli-derived plasmid template containing DNA coding GLuc, thereby inducing site-specific mutation. (ii) DpnIenzyme treatment, which enables decomposition of only E-coli-derived plasmid, is performed to decompose the plasmid used as the template contained in the PCR product. (iii) The presence or absence of mutation is confirmed by performing gene sequence assay.

For the template DNA encoding GLuc, the base sequence (GENE ACCESSION #: FJ010198) registered in a known database may be used without modification, or with partial modification. Here, as stated below in Section 3, it is possible to use a modified DNA for further improvement of the function as a template DNA for the Quick-Change method. To synthesize the DNA encoding the GLuc or a modified DNA, a set of multiple primers are prepared first. Then, the primers are set to the sense and antisense positions to be subjected to PCR reaction, thereby extending the two fragments to create a DNA template containing the entire length of Gaussia luciferase gene. This template may be mass-produced by, for example, subcloning the obtained DNA template into eukaryotic expression vectors pcDNA 3.1(+) and multiplying them in procaryotic cells such as Escherichia coli.

### (2-3) Modification Strategy for Improving Optical Properties

To determine the replacement sites of amino acid residues in marine luciferase, the present inventors first focused attention on the following points.
(i) According to a hydrophobic sequence assay of the entire amino acid sequence of GLuc, there is a hydrophilic amino acid region in the central region of the enzyme (FIG. 4B). The hydrophilic region in the central region of a protein denotes that the portion is externally exposed. Therefore, the inventors presumed that the portion is a region to which the substrate can easily access; more specifically, the portion is an interaction site with the substrate, i.e., the region includes the active core of the enzyme.
(ii) According to a report (Non-patent Literature 15) regarding the crystal analysis of Renilla-derived luciferase (Renilla luciferase; RLuc), which is a marine luciferase, a hydrophobic amino acid, such as isoleucine (Ile), is present at regular intervals in the vicinity of the hydrophilic region, which is the presumed enzyme active site (as explained in (i)). Since such regular appearance of hydrophobic amino acids is also observed in GLuc, the inventors speculated that the region near the isoleucine (Ile) repeat sequence is the critical area of enzymatic activity where the interaction with the substrate takes place.
(iii) In the past, the present inventors conducted a series of divisional studies in which six portions of the central region of GLuc are cleaved, and thereby found that the regions in the vicinity of amino acids No. 93 and 98 are the critical area of enzymatic activity (Non-patent Literature 14) (FIG. 3A). This finding is consistent with the findings described in (i) and (ii) above.
(iv) Another clue is obtained by the overlapping fashion in the single sequence of marine luciferases, i.e., a Single-Sequence Alignment (SSA). The arrowheads in FIG. 17 suggest putative mutation sites to increase consensus amino acids, i.e., amino acids No. 90 to 100. For example, I90 corresponds with L161 in the SSA. The substitution of the isoleucine with leucine (I90L) interestingly exerted a great increase in the optical intensity as shown in the examples.

Following a thorough study of the above findings (i) to (iv), the inventors speculated that the area corresponding to 74 to 131 of GLuc among the central region of the amino acid sequence of marine luciferase is the active center of enzyme. The inventors further speculated that the area near the hydrophobic amino acid repeat sequence is the portion where the interaction with the substrate actually takes place.

Therefore, in the following, point mutation is introduced mainly into hydrophobic amino acids at positions 74 to 131 in the GLuc sequence.

The amino acids to be replaced were selected according to the three hypothesized interactions with the substrate in the active center, i.e., (a) a hydrogen bond with the substrate, (b) an aromatic interaction with the phenyl group of the substrate, and (c) an interaction between respective hydrophobic residues of the substrate and the enzyme. The inventors hypothesized that the enzyme active group could be improved by adjusting these three interactions.

Further, in the replacement of amino acid residues, the inventors set the major condition “conservative amino acid replacement,” in which the candidate is selected from a group of similar amino acid residues, so as to avoid changes in the environment of the enzyme active center where enzymatic activity takes place.

More specifically, the amino acids that presumably form a hydrogen bond with the substrate as stated in (a) are amino acids irrelevant to the peptide bond, or polar amino acids having carboxy group, amide group or hydroxyl group; namely, neutral amino acids such as asparagine, serine, threonine, glutamine, cysteine and the like; acidic amino acids such as asparagic acid, glutamic acid and the like; and basic amino acids such as arginine, histidine,lysine and the like. Among these amino acids, typical neutral amino acids are asparagine, threonine, serine and glutamine, a typical acidic amino acid is glutamic acid, and a typical basic amino acid is histidine. Such amino acids are used upon replacement of amino acids at at least one position selected from positions 90, 93 to 95, 97, 99, and 100.

Specific examples of amino acids capable of aromatic interaction with the phenyl group of the substrate as stated in (b) include aromatic amino acids or heterocyclic amino acids having a n-electron, namely, phenylalanine (Phe), thyrosine (Tyr), tryptophan (Trp), and histidine (His). Using these amino acids, the amino acids at least one position selected from positions 90, 93 to 95, 97 and 130 are replaced. Among them, thyrosine (Tyr) and histidine (His) belong to the aforementioned amino acid group (a), and phenylalanine (Phe) and tryptophan (Trp) belong to the amino acid group (c).

Specific examples of amino acids capable of interaction between respective hydrophobic residues of the substrate and the enzyme as stated in (c) are hydrophobic nonpolar amino acid residues, namely, Leu (leucine), Ile (isoleucine), Val (valine), Ala (alanine), Phe (phenylalanine), Pro (proline), Met (methionine), Trp (tryptophan), and Gly (glycine). Typical examples of these amino acids are Leu (leucine), Ile (isoleucine), Val (valine), phenylalanine (Phe), tryptophan (Trp), and glycine (Gly). Such amino acids are used to replace the amino acids at at least one position selected from positions 74, 77, 90, 93 to 95, 97, 108, 112, 115, 118, 121, 130, and 131. Phenylalanine (Phe) and tryptophan (Trp) belong to the aforementioned amino acid group (b).

(2-4) Luciferase Variant of the Present Invention with Improved Optical Properties

According to the results of the experiment based on the aforementioned strategies (i), (ii) and (iii), improvement in optical properties was observed in amino acids having mutations at positions 89 to 118 among the amino acids 74 to 131 of GLuc. More specifically, conservative amino acid replacement was performed with respect to an amino acid at at least one position selected from position 89, 90, 95, 97, 100, 108, 112, 115, or 118, thereby enhancing the bioluminescence of luciferase and/or stabilizing the bioluminescence intensity, and/or exerting a red-shift. Thus, improvement in optical properties of luminescence enzyme was accomplished.

The replacement that rendered the greatest improvement was a conservative amino acid replacement at position 90, 108, 112, 115, or 118 based on the aforementioned strategy (iii), or a conservative amino acid replacement at position 89 or 97 based on the aforementioned strategy (ii). The replacement enhanced the intensity of luciferase, stabilized the luminescence, and admitted a red-shift of spectra. More specifically, the replacement of the hydrophobic and nonpolar (in particular, aliphatic) amino acid residue (more specifically, isoleucine) at least one position selected from positions 90, 108, 112, 115, and 118 with other hydrophobic and nonpolar (in particular, aliphatic) amino acid residue (more specifically, leucine, tryptophan, or valine); and replacement of the aromatic amino acid residues at least one position selected from position 89 and (more specifically, phenylalanine at position 89 and/or thyrosine at position 97) with other aromatic amino acid residue (more specifically, tryptophan) particularly enhanced and stabilized the luminescence intensity, while exerting a red-shift. In particular, the variant (I90L) obtained by replacing isoleucine at position 90 with leucine was a superior luminescent enzyme variant having significantly high and stable luminescence intensity, and a shift to a long wavelength side.

The replacement of leucine at position 90 with thyrosine is also regarded as a replacement between hydrophobic amino acid residues, and therefore may also be regarded as a conservative amino acid replacement. This replacement admitted a red-shift, even though no enhancement in luminescence intensity was observed. According to the fact that the replacement of phenyl alanine at position 89 and thyrosine at position 97 with tryptophan are also considered replacement between hydrophobic amino acid residues, the above effect can also be expressed such that replacement of hydrophobic amino acid residues at at least one position selected from positions 89, 90, 97, 108, 112, 115, and 118 in GLuc respectively with another hydrophobic amino acid residue improves activity of a luciferase; such improvement is specifically substantiated by enhancement and stabilization of bioluminescence intensity and/or a shift to longer wavelength. Further, since the replacement of a hydrophilic amino acid residue at position 95 and/or 100 that forms a hydrogen bond with another hydrophilic amino acid residue (more specifically, replacement of histidine at position 95 with glutamic acid, and/or replacement of asparagic acid at position 100 with asparagine) exerts a red-shift, it was confirmed that the aforementioned strategy (i) is also effective to admit a red-shift. A red-shift is equivalent to an enhancement in light transmittance in the observation through cell membrane or skin. Therefore, this is a desired characteristic.

Furthermore, it was also confirmed that the optical property was further improved by introducing multiple mutations according to the aforementioned strategies (i), (ii) and (iii) instead of introducing only one mutation into one of the above positions. More specifically, in the present invention, it is also effective to introduce mutations into multiple sites at the same time. For example, by combining the mutation for enhancing bioluminescence intensity and for improving bioluminescence stability (more specifically, the mutation of at least one position of 89, 90, 108, 112, 115, and 118 in the amino acid sequence of GLuc with a hydrophobic amino acid or acids) with the mutation for exerting a red-shift(more specifically, the mutation of at least one position of 90, 95, 97 and 100), it becomes possible to further enhance the luminescence intensity, improve luminescence stability, and exert a red-shift.

More specifically, both the modified luciferase (F89W/I90L) obtained by replacing isoleucine (I) at position 90 in the amino acid sequence of GLuc with leucine (L), and replacing phenylalanine (F) at position 89 with tryptophan (W); and a modified luciferase (I90L/I115L) obtained by replacing isoleucine (I) at position 90 with leucine (L), and replacing isoleucine (I) at position 115 with leucine (L) are revolutionary luciferases that have very high and stable luminescence intensity, and cause a shift to longer wavelength.

In order to obtain a variant with further improved optical property, a modified luciferase having mutations at “F89W/I90L” can be further mutated by replacing histidine (H) at position 95 with glutamic acid (E) or glutamine (Q), and replacing thyrosine (Y) at position 97 with tryptophan (W) (F89W/I90L/H95E/Y97W); and a further shift to long wavelength is observed while maintaining the improved bioluminescence intensity and stability. In particular, the modified luciferase “F89W/I90L/H95E/Y97W” shows a great shift to a long wavelength side, which was shown as an increase of the peak value from 471 nm (absorbency), which indicates the maximum bioluminescence intensity of the original GLuc, to 506 nm (absorbency).

The stability of the bioluminescence intensity is confirmed by introducing a substrate (dissolved in PBS buffer) into each variant, and observing its changes in bioluminescence with time. As a result of a 5-second observation of I90L, H95E, and Y97W that are typical GLuc variants, it was found that they had stable luminescence intensities (FIG. 7A). Further observation (10 minutes) revealed that, whereas the decrease in luminescence intensity of the original GLuc (white circle) was significant, the luminescence intensity of I90L (black square) and the luminescence intensity of I90V (gray triangle) were relatively stable (FIG. 7B). The same result was obtained from a short-time (5 seconds) observation of each variant for its changes in bioluminescence with time after introducing a substrate (dissolved in a modified Matthew's buffer) (FIG. 7C). Furthermore, another observation of 1 minute for each stage of changes in bioluminescence with time after introducing a substrate (dissolved in modified Matthew buffer) stepwise into each variant also revealed that each variant had very stable and strong bioluminescence (FIG. 7D).

### (2-5) Other Gaussia-Like Luciferase Variants

Using a Gaussia-like luciferase derived from a Copepod (Metridia pacifica or Metridia longa), which is a Gaussia-like marine animal very similar to Gaussia, namely, luciferases of MpLuc1 (derived from Metridia pacifica) and MLuc (derived from Metridia longa), a MLuc-I123L variant and a MpLuc1-I114L variant can be obtained by carrying out conservative amino acid replacements, for example, of an amino acid at position 123 of MLuc and an amino acid at position 114 of MpLuc1 corresponding to the amino acid at position 90 of GLuc. Further examples include a Y122W/I123L/H128E/Y130W variant (MLuc4) of MLuc and a Y113W/I114L/H119E/Y121W variant (MpLuc4) of MpLuc1 corresponding to a F89W/I90L/H95E/Y97W variant (Mon3) of GLuc. The luminescence characteristics of these variants were measured, with the result that their bioluminescence intensities were enhanced as in the GLuc variant. The results also revealed an enhancement in stability and a red-shifted property (FIG. 14A to 14C and FIG. 15A to 15D). This confirmed that the findings regarding GLuc are also applicable to all of the other Gaussia-like luciferases.

(2-6) Luciferase Variant with Improved Luminescent Function Used for the Present Invention

The above mentioned marine luciferase of the present invention, in particular, the Gaussia-like luciferase, is obtained by “conservative amino acid replacement” of at least one amino acid residue selected from the amino acid residues at least one position selected from positions 89, 90, 95, 97, 100, 108, 112, 115, and 118 among the amino acid sequence at positions 89 to 118 of Gaussia luciferase (GLuc). More specifically, such replacement includes replacement of a hydrophobic amino acid residue at least one position selected from positions 89, 90, 97, 108, 112, 115, and 118 in the amino acid sequence of GLuc with another hydrophobic amino acid residue, and/or replacement of a hydrophilic amino acid residues at least one position selected from positions 95 and 100 that forms a hydrogen bond with another hydrophilic amino acid residue.

This hydrophobic amino acid is preferably a hydrophobic amino acid having a greater molecular weight than a small-size hydrophobic amino acid such as glycine (Gly). An aromatic amino acid, such as tryptophan (Trp), may be used insofar as it is hydrophobic and nonpolar. In particular, among the hydrophobic amino acids, aliphatic amino acids, such as leucine (Leu), valine (Val), isoleucine (Ile) or tryptophan (Trp), are preferable. Lleucine (Leu), valine (Val), and tryptophan (Trp) are particularly preferable.

Further, when the replacement of a hydrophobic amino acid residue with another hydrophobic amino acid residue is replacement of a hydrophobic nonpolar aliphatic amino acid residue at least one position selected from positions 90, 108, 112, 115, and 118 in the amino acid sequence of GLuc with another hydrophobic nonpolar aliphatic amino acid residue (in particular, the replacement of isoleucine with one amino acid residue selected from leucine, tryptophan and valine), and/or replacement of a hydrophobic aromatic amino residue at least one position selected from positions 89 and 97 with another hydrophobic aromatic amino residue (the replacement of phenylalanine (Phe) at position 89 and/or thyrosine (Tyr) at positions 97 with tryptophan (Trp)), the resulting luciferase variant has an improved luminescence intensity, as well as stable luminescence intensity or red-shifted wavelength. Thus, the enhancement in activity of luminescence enzyme can be accomplished. The most preferable combination of the mutation position and the mutating amino acid for such replacement is replacement of position 89 with tryptophan (Trp), replacement of position 90 with Leu (leucine) or Val (valine), and replacement of position 108 with Val (valine).

Further, when the replacement of a hydrophilic amino acid residue that forms a hydrogen bond with another hydrophilic amino acid residue is replacement of histidine (His) at position 95 in the amino acid sequence of GLuc with glutamic acid (Glu), and/or replacement of asparagic acid (Asp) at position 100 with asparagine (Asn), such a replacement causes a shift to longer wavelength of the resulting modified luciferase.

Among the modified luciferases, the modified luciferases (GLuc-I90L, MpLuc1-I114L, MpLuc1-I114L) having a mutation caused by replacement of isoleucine corresponding to position 90 in the amino acid sequence of GLuc is preferred in view of the high intensity, and improved stability, and red-shifted wavelength. It is more preferable to produce a variant by at least one of the following: replacing an amino acid corresponding to position 89 in the amino acid sequence of GLuc with tryptophan, replacing an amino acid corresponding to position 90 with leucine or valine, replacing an amino acid at position 95 with glutamic acid, replacing an amino acid at position 97 with tryptophan, replacing an amino acid at position 108 with valine, replacing an amino acid at position 112 with leucine or valine, replacing an amino acid at position 115 with valine, and replacing an amino acid at position 118 with leucine or valine, so as to further significantly enhance luminescence intensity and cause a shift to longer wavelength. In particular, the leucine variants (GLuc-I90L, MpLuc1-I114L, MpLuc1-I114L) having mutation at position 90 are excellent modified luciferases with not only a high luminescence intensity, but also a significant red-shifted wavelength.

In the present invention, “a luciferase variant (modified luciferase)” preferably denotes a variant basically having no mutation with respect to the amino acid sequence of the original Gaussia luciferase other than the aforementioned “conservative amino acid replacement”, in the region corresponding to positions 89 to 118, i.e., in the active center of the amino acid sequence. However, as long as the specified region has no mutation other than the conservative amino acid replacement, the variant may have mutation in other positions on the condition that the muta:ion is not conducive to a severe change in the entire insteric structure. In particular, the variant may contain a deletion at a part of the N- or C-terminal region, for example, deletion of 1 to 50, preferably 1 to 30, more preferably 1 to 20, further preferably 1 to 10 amino acid residues in the N- or C-terminal region. More specifically, the variant may mean a modified Gaussia-like luciferase having an amino acid sequence with homology of 70% or more, preferably 80% or more, more preferably 90% or more, most preferably 95%, except for the region corresponding to positions 89 to 118 in the amino acid sequence of GLuc.

### 3. Change of Marine Luciferase Gene of the Present Invention to Optimal Codons for Mammalian

The usages of the marine luciferase of the present invention are described in Section 4 below. Typically, the usages include those as a luciferase component of a known bioluminescent probe, and, owing to its high luminance, as a substitute for a reporter gene for fluorescent imaging in vivo. Since analysis methods using reporter genes, such as the reporter gene assay or the two-hybrid method, are performed in the hitherto-established host cell systems, the series of procedures of producing a vector by fusing the luciferase variant gene of the present invention to the promoter of the target gene, and culturing the host cell incorporating the vector may be performed according to well-known methods. Examples of host cells used herein include yeast cells, bacteria cells such as Escherichia coli, and insect cells, as well as mammalian cells (COS cell, CHO-K1 cell, HeLa cell, HEK293 cell, NIH3T3 cell) used for general gene recombination. The present invention is mainly used in mammalian cells, such as humans, in vivo or in vitro.

Further, the luciferase variant gene of the present invention is preferably modified to have codons suitable for the target cell, thereby modifying the base sequence into a sequence that is more easily expressed in the host cell. The luciferase variant gene of the present invention also needs a modification to contain a restriction enzyme site to be introduced into a vector. These modifications may also be performed according to known methods. As one example, a modification strategy of a variant assumed to be used in mammalian cells, such as humans in vivo or in mammalian cells in vitro, is described below.

More specifically, the modifications for improving other functions include modification of the codons corresponding to the amino acid into those suitable for mammalians so that the gene has a base sequence more easily expressed in the bodies of mammalian such as humans, and a modification to introduce a restriction enzyme site at a desired position. Accordingly, the present invention adopts such modifications as required. The modification strategies for improving functions are specifically described below. However, the present invention is not limited to these examples.
(i) The modification was conducted by taking into consideration the fact that organisms have. a common molecular mechanism for expressing the same DNA, bu: may have a different gene codon proportion depending on the species. For example, an animal cell has a higher proportion of G or C in the nucleic acid than that in Escherichia coli, or tends to express genes with a higher proportion of G and C. Therefore, in order to ensure effective expression of GLuc in an animal cell, the known gene sequence of the GLuc is modified by optimizing the codons to have a higher proportion of G or C.
(ii) The Kozak sequence (GCCACCATGG) is introduced in the first position of the gene so as to ensure high expression in eukaryotic cells.

(iii) The original extracellular secretion signal (1-17AA) on the upstream of GLuc gene, which is irrelevant to the luminescence activity, is deleted or replaced with another known extracellular secretion signal or intracellular signal. Alternatively, an additional intracellular signal may be provided in the C-terminal end while leaving the secretion signal as is.

In the embodiments of the present invention, a template of DNA encoding GLuc was designed according to the sequence information of GLuc, in consideration of the above points (i) to (iii). A primer set for synthesizing the DNA was constructed, and the template of GLuc gene was completed by PCR reaction. The template was ligated into pcDNA 3.1(+) of a eukaryotic cell expression vector to carry out subcloning.

Using the above expression vector as a template, a point mutation was introduced according to the aforementioned “Quick-Change method.” More specifically, a PCR reaction was performed by coexisting a sense primer and an antisense primer of about 30 bases prepared by containing a mutation point, thereby inducing site-specific mutation. Then, only the E. coli-derived plasmid containing the GLuc gene template was decomposed by DpnIenzyme, and the gene sequence was analyzed to confirm the presence or absence of mutation.

### 4. Method for Confirming Enzymatic Activity of Mutant Enzyme of the Present Invention

The enzymatic activity of mutated GLuc may be verified, for example, in the following manner.

First, using a known lipid reagent for gene introduction, an expression vector having a GLuc variant is introduced into African monkey cells (COS-7); while also introducing, as a control, an expression vector having a known unmodified GLuc without any mutation into the cells in the same manner. At a predetermined time (from 10 to 20 hours, for example, 16 hours) after the introduction of the vector, a cell lysate is prepared using a known lysis solution.

Thereafter, the cell lysate is mixed with a known substrate solution containing coelenterazine, and its color intensity, temporal stability in luminescence, etc., are measured.

The luminescence intensity may be found by measuring the intensity at a specific wavelength using a known luminescence spectrophotometer after addition of a known substrate. By performing the measurement every minute, the temporal stability in luminescence can be found. To measure a shift to longer wavelength, scanning of the entire wavelength is necessary.

In the embodiments of the present invention, the variant obtained by replacing hydrophobic amino acids at one or more positions selected from position 89, 90, 108, 112, 115, and 118 in GLuc with other hydrophobic amino acids had enhanced luminescence intensity. More specifically, a variant obtained by replacing the amino acid 89 with tryptophan, 90 with leucineor valine, 108 with valine, 112 with leucineor valine, 115 with valine, and 118 with leucineor valine showed an intense luminescence spectrum peak (luminescence spectrophotometer). In the measurement of the area of luminescence spectrum using a luminescence plate reader, the glutamic acid variant having a mutation at position 95 and the tryptophan variant having a mutation at position 97 showed a significant enhancement in luminescence intensity, as well as the leucine variant having a mutation at position 90; further, these variants all caused a shift to longer wavelength. In particular, the leucine variant (I90L) having a mutation at position 90 is a superior luciferase variant that has a high luminance and a significant shift to longer wavelength. Further, the variant with two simultaneous mutations (F89W/I90L or I90L/I115L), i.e., the leucine replacement at position 90, and tryptophan replacement at position 89 or leucine replacement at position 115 at the same time; and the variant with four simultaneous mutations (F89W/I90L/H95E/Y97W), i.e., leucine replacement at position 90, tryptophan replacement at position 89, glutamic acid replacement at position 95, and tryptophan replacement at position 97 at the same time, both showed a significant luminescence intensity and a significant shift to a long wavelength side. Thus, they both were revolutionary luciferase variants.

Further, the variant having replacement with glutamine (Gln) or asparagine (Asn), which is a neutral amino acid forming a hydrogen bond, at position 95 or 100 showed a shift of bioluminescence to long wavelength. Similarly, it was confirmed that the variants having mutations at positions 90 and 97 showed a shift of bioluminescence to a long wavelength side, regardless of whether the mutation introduces leucine (Leu), which is a hydrophobic amino acid, thyrosine (Tyr), which is an amino acid forming a hydrogen bond and also an aromatic amino acid, or tryptophan (Trp), which is an aromatic amino acid and also a hydrophobic amino acid. The shift to longer wavelength was substantiated by the luminescence peaks of variant Y97W and variant I90Y, which were 486 nm and 492 nm, respectively, relative to the luminescence peak of GLuc, which was 471 nm. Further, the variants I90L, F39W/I90L, and I90L/I115L also showed significant shifts to a long wavelength side, as they had luminescence peaks of 486 nm, 481 nm, and 483 nm, respectively. In particular, the luminescence peak of the variant F89W/I90L/H95E/Y97W was 506 nm, showing a revolutionary shift to longer wavelength. Such a long wavelength bioluminescence improved transmittance through living tissues, and is thereby highly useful as a valuable analytical signal.

### 5. Use of the Present Invention as Modified Luciferase Gene

The gene of the present invention encoding a luciferase variant is most expected to be used as a reporter gene for various analysis systems, as with the conventional luciferase genes, and as a bioluminescent probe that is currently under research and development.

### (5-1) Reporter Gene Assay

The marine luciferase variant of the present invention may be used as the widely known reporter gene assay (reporter gene assay), yeast two-hybrid assay, mammalian two-hybrid assay, protein splicing assay, protein complementation assay, circular permutation assay, bioluminescence resonance energy transfer assay (BRET) or the like as a core element, and is conducive to significant improvement in measurement performance in these assays. The marine luciferase variant of the present invention can completely overcome the following drawbacks of conventional firefly luciferase:
(i) Due to its large molecular weight, it takes a long time to start expression, thereby imposing a great burden on the host cells.
(ii) Due to the low luminescence intensity of firefly luciferase, it generally takes 1 to 2 days after stimulation to obtain a sufficient accumulation of luciferase (reporter).

Since the use of the superluminescent luciferase of the present invention ensures a significantly high luminescence intensity of the reporter (in particular, the variant I90L has a luminance 14000 times greater than that of a known firefly luciferase), it has an advantage of very prompt measurement after the stimulation. Accordingly, the measurement time can be greatly reduced while ensuring high temporal stability in luminescence, thereby enabling luminescence measurement even for a cell strain with insufficient gene intrcduction. Further, the red-shifted luminescence improves transmittance through cell membrane or skin, thereby ensuring high measurement sensitivity.

More specifically, the superluminescent luciferase of the present invention is employed in these reporter gene assays in such a manner that the luciferase is linked to a known eukaryotic cell expression vector containing a special promoter in an upsteam portion, and the vector is then introduced into a eukaryotic cell. After a predetermined time, the measurement is performed using the luciferase either in the presence or absence of signal (stimulation) (Non-patent Literature 13). A known p-TransLucent vector can be used as this expression vector for reporter gene assay that can carry the luciferase of the present invention; the luciferase can easily be incorporated therein using a known method. The commercially available pG5Luc vector can be used as this vector for the two-hybrid assay; the luciferase can easily be incorporated therein using a known method.

### (5-2) Application to Luminescent Probe

Further, by incorporating the superluminescent luciferase of the present invention into the single-molecule-format luminescent probe (Non-patent Literature 9, Non-patent Literature 10, Non-patent Literature 11, Non-patent Literature 12, Japanese Patent Application No. 2007-005144, 2007-202308, 2007-332253, 2008-116098, and 2009-025229, PCT/JP2008/050370, U.S. Ser. No. 12/025,532, U.S. Ser. No. 12/343,830 which are incorporated herein by reference in its entirety), or the two-molecule-format luminescent probe (Non-patent Literature 4, Non-patent Literature 6, and Non-patent Literature 7, which are incorporated herein by reference in its entirety) which are recited in the pending patents applied by the present inventors, the presence or absence of ligand and the intensity of ligand activity can be observed with high luminance. By comprising, as the probe components, (i) the dissected luciferase (N- and C-terminal fragments), and (ii) a ligand-binding protein responsive to the target ligand and (iii) a recognition protein that recognizes the bond of the ligand with the ligand-binding protein, which are linked to the vicinity of the dissected luciferase, it is possible to form a high-performance luminescent probe. This luminescent probe functions such that, as the recognition protein recognizes the ligand binding of the ligand-binding protein, the two adjacent fragments of the dissected enzyme complement each other and thereby change the enzymatic activity. Here, due to the high luminance and stability of the dissected enzyme, it is possible to perform reliable measurement with an improved detection limit.

In the present invention, “single molecule-format luminescent probe” denotes a known bioluminescent probe in which all components for visualization imaging are integrated in a single fusion molecule (disclosed in Patent Literature 7, etc.). For example, “single molecule-format luminescent probe” denotes a fusion protein that comprises, as fundamental components, the two fragments of N- and C-terminals obtained by dissecting the marine luciferase variant of the present invention, a ligand-binding protein, and a recognition protein for recognizing the ligand-binding protein. Similarly, “two molecule-format luminescent probe” in the present invention denotes a bioluminescent probe in which the two fragments of N- and C-terminals obtained by dissecting the marine luciferase variant of the present invention are present in the fusion protein containing the ligand-binding protein, and in the fusion protein containing the recognition protein, respectively.

As such, when the marine luciferase superluminant variant of the present invention is used for these bioluminescent probes, the variant must be dissected into a N-terminal fragment and a C-terminal fragment. The dissection portion is the same as the dissection portion of the marine luciferase used for the known “single molecule-format luminescent probe” and “two molecule-format luminescent probes”.

More specifically, the GLuc variant of the present invention is dissected at a portion between 105 and 110, and the MLuc variant of the present invention is dissected at a portion between 138 and 142.

Further, in addition to the single molecule-format bioluminescent probe, the superluminant luciferase may also be incorporated in the circular permutation replacement probe (Non-patent Literature 11; Non-patent Literature 12) developed by the present inventors, thereby enabling efficient calculation of intracellular molecular phenomenon with stable and high luminescence intensity. In addition to 8990N and Mon3N, i.e., the single molecule-format bioluminescent probes described in Example 9 of the present invention, the circularly permutated versions of the probes are cPresso and cPressoMax in the sequence list.

Regarding the actual method for using the superluminescent luciferase of the present invention as a single molecule-format luminescent probe, Patent Literature 7, and Japanese Patent Application Nos. 2007-332253 and 2009-025229 disclose the details thereof. More specifically, the marine luciferase of the present invention is dissected, and a chimera DNA encoding a luminescent probe in which a ligand-binding protein and a peptide sequence, which recognizes the change in steric structure upon binding of a ligand to the protein, are linked in a linear chain form. Generally, the chimera DNA is subcloned into a vector suitable for the cells in which the chimera DNA is intended to be expressed, and the vector is introduced into the cells to be expressed. However, the chimera DNA may be ligated to a control sequence at an upstream portion to he directly introduced into the cells. The target cells are preferably mammalian-derived cells such as human cells. Other suitable examples include cells that exist in a living subject, and culture cells that retain the native function, yeast cells, insect cells, and procaryotic cells such as Escherichia coli. The type of the vector is also not particularly limited. A suitable vector capable of being expressed in the target host cells is appropriately selected. The introduction of the vector into the cells are performed using known transfection methods such as a microinjection method or an electroporation method, or a transfection method using a lipid reagent (BioPORTER (Gene Therapy Systems, Inc.), Chariot (Active Motif), etc.).

Since the bioluminescent probe using the superluminescent luciferase of the present invention is introduced into cells as a chimera DNA and expressed in the cells as a fusion protein, by measuring the change in light amount emitted from the cells after subjecting the transformed cell to ligand stimulation, the property or levels of activity of the ligand may be evaluated.

When the superluminescent luciferase of the present invention is incorporated in the bioluminescent probe, the “ligand-binding protein,” which can be incorporated in the probe together with the luciferase, is intended to mean a protein that binds with a ligand at the ligand binding site. The ligand-binding protein may serve to, in response to the bond with the ligand, for example, change the steric structure, cause phosphorylation, or facilitate protein-protein interaction. Examples of such ligand-binding proteins include nuclear receptors (NR) to which such ligands as hormones, chemical substances, or signal transduction proteins bind; cytokine receptors; and various protein kinases. A suitable ligand-binding protein is selected depending on the target ligand. The ligand that binds to the ligand-binding protein is not particularly limited insofar as it binds to the ligand-binding protein. The ligand may be an extracellular ligand that is introduced in response to extracellular stimulus, or an intracellular ligand that is produced inside the cells. Examples thereof include agonists or antagonists of the receptor protein (for example, intranuclear receptor, or G-protein-linked receptor), signal transduction proteins such as cytokine, chemokine, or insulin, intracellular second messenger, lipid second messenger, phosphorylated amino acid residue, G-protein-linked receptor ligand and like ligands that specifically bind to proteins involved in intracellular signal transduction.

For example, when the intracellular second messenger, the lipid second messenger or the like is used as a ligand, the binding domain of each second messenger may be used as the ligand-binding protein. “Second messenger” denotes a different kind of intracellular signal transduction substance that is newly produced as a result of the bond of the extracellular signal transduction substance, such as a hormone or neurotransmission substance, with a receptor that exists in the cell membrane.

Examples of the second messengers include cGMP, AMP, PIP, PIP₂, PIP₃, inositol trisphosphate (IP₃), IP₄, Ca²⁺, diacylglycerol, and arachidonic achid. For example, for Ca2+ as the second messenger, calmodulin (CaM) may be used as the ligand-binding protein.

### (5-3) Intracellular Imaging

Further, using the gene encoding the superluminescent luciferase enables stable introduction of the luciferase into various cell strains. For example, using the gene enables stable introduction of the luciferase into the novel induced pluripotent stem cells (iPS cells). Since the induced pluripotent stem cells (iPS) do not emit light themselves, it has been very difficult to research the intracellular molecular phenomenon and tissue specificity of the cells. To address this difficulty, a molecular probe containing the luciferase is introduced into somatic cells before the embryo is formed; after the introduction of the luciferase-containing probe, the embryo is differentiated into various tissues. This enables measurement of specific molecular phenomena in respective organs at high sensitivity.

This process is performed according to the method of Yamanaka et al. (Non-patent Literature 19).

Further, by linking the superluminescent luciferase of the present invention to a suitable signal peptide, the luciferase can be used for luminance imaging of various organelles. For example, by linking a GAP-43-derived MLCCMRRTKQV sequence to an N- or C-terminal of GLuc, the luciferase may be localized in cell membranes. Linking a GRKKRRQRRR sequence to a terminal enables localization in cell cytoplasm. Further, for localization in endoplasmic reticulum (ER) and cellular nucleus, KDEL and DPKKKRKV sequences, respectively, are linked to a terminal. Furthermore, by linking to HIS-tag (HHHHHH), FLAG-tag (DYKDDDDK), Myc-tag (EQKLISEEDL), HA-tag (YPYDVPDYA), V5-tag (GKPIPNPLLGLDST), T7-tag (MASMTGGQQMG) or like antigen sites, the luciferase can be used for immunostaining or separation/refinement in acellular systems. In these usages, known immunostaining technologies or immunocytochemistry may be adopted.

### 6. Other Remarks

The other terms and concepts used in the present invention are specifically defined in the descriptions of embodiments and examples. The terms are generally selected from the IUPAC-IUB Commission on Biochemical Nomenclature, or based on the interpretations of idiomatic terms and words in the related field. Further, except for the technologies with apparent sources, the various technologies used to carry out the present invention can be easily and consistently performed by persons skilled in the art in reference to published documents, etc. For example, genetic engineering and molecular biological techniques can be carried out according to J. Sambrook, E. F. Fritsch & T. Maniatis, “Molecular Cloning: A Laboratory Manual (2nd edition),” Cold Spring Harbor Laboratory Press, Cold Spring Harbor, N.Y. (1989); D. M. Glover et al. ed., “DNA Cloning,” 2nd ed., Vols. 1 to 4, (The Practical Approach Series), IRL Press, Oxford University Press (1995); Ausubel, F. M. et al., Current Protocols in Molecular Biology, John Wiley & Sons, New York, N.Y., 1995; “Zoku Seikagaku Jikken Koza 1 (Continuation of Biochemistry Experimental Series 1), Idensi Kenkyu Ho (Gene Study Method) II” edited by Tokyo Kagaku Dojin (1986); “Shin Seikagaku Jikken Koza 2 (New Biochemistry Experimental Series 2), Kakusan (Nucleic Acid) III (DNA Recombinant Technology)” edited by Tokyo Kagaku Dojin (1986); Japanese Biochemical Society, Tokyo Kagaku Dojin (1992); R. Wu ed., “Methods in Enzymology,” Vol. 68 (Recombinant DNA), Academic Press, New York (1980); R. Wu et al. ed., “Methods in Enzymology,” Vols. 100 (Recombinant DNA, Part B) & 101 (Recombinant DNA, Part C), Academic Press, New York (1983); R. Wu et al. ed., “Methods in Enzymology,” Vols. 153 (Recombinant DNA, Part D), 154 (Recombinant DNA, Part E) & 155 (Recombinant DNA, Part F), Academic Press, New York (1987), etc.; the methods mentioned in the documents referenced in these documents, or other various similar methods and modified methods thereof that are substantially the same as the disclosed methods.

Further, the various proteins, peptides, and DNAs encoding them used in the present invention may be obtained from existing databases (URL: http://www.ncbi.nlm.nih.gov, etc.).

### EXAMPLES

Hereinbelow, the present invention is specifically described in detail with reference to examples; however, the present invention is not limited to these examples.

Note that the disclosures of the technical documents, patent publications, and patent application specifications are incorporated into the specification by reference.

### Example 1

### Construction of Gene Sequence Encoding GLuc

In the determination of a new gene sequence based on a known gene sequence (GENE ACCESSION #: FJ010198) of Gaussia luciferase (GLuc), codons were optimized for known mammalian cells. Specifically, codons that contain many glycines and cysteines must be produced for the optimal expression in mammalian cells. For example, of two base codons, TTT and TTC, encoding phenylalanine among amino acids, TTC is more suitable for mammalian cells. Additionally, in order to increase the protein expression levels, a known Kozak sequence (GCCRCCATGG) was inserted before and after the translation initiation codon (AUG).

In order to generate the above gene sequence, sense and antisense primers were palindromically placed, and a PCR reaction was performed to synthesize the full-length sequence. The full-length GLuc was used as a genetic template upon introduction of other mutations in native GLuc. FIG. 2 shows the full length.

### Example 2

### Search for the Enzyme Active Site in GLuc and Introduction of Mutations

(2-1) The following inferences were made in the introduction of a mutation into native Gaussia Luciferase (GLuc).

Inference 1: Through a detailed observation of the crystallographic structure (FIG. 3C) formed by Renilla luciferase (RLuc) upon binding coelenterazine, which is a common substrate of marine luciferases, it was found that the active site of RLuc showed following properties:
(1) there is a region, where hydrophobic amino acids are found at certain intervals ranging Erom 2 to 4 amino acid residues, and
(2) the active site has a structure in which the substrate is sandwiched between hydrophilic and hydrophobic amino acid sites (FIG. 2B).

Through an examination of GLuc amino acid sequence, it was found that, in the region at a similar site (amino acid numbers 74-161), hydrophobic amino acids (isoleucine (I), leucine (L), valine (V), phenylalanine (F), tryptophan (W), and the like) appear at certain intervals ranging from 2 to 4 amino acid residues; and it was inferred that, also in GLuc, this region is the substrate-binding region, which is the key site that influences luciferase activity.

Inference 2: First, in order to estimate the active site in the luciferase, several sites in the center of the enzyme were fragmented, and calmodulin (CaM) was then inserted between the fragments, thereby examining whether the enzymatic activity is reconstituted. As a result, hardly any enzymatic activity was observed at dissection sites #1-#3. On the other hand, a very strong bioluminescence was observed at dissection site #4 (FIG. 3A).

It was confirmed from the study on the dissection sites in GLuc that the amino acid sequences near the dissection sites #3 and #4 are the key regions in GLuc that influence the enzyme activity of GLuc.

Inference 3: Further, the hydrophobic sequence search of GLuc revealed that the above site is the region of intersection of hydrophilic and hydrophobic amino acid domains (FIG. 3B).

Inference 4: Conventional studies on the chromophores of fluorescent proteins were researched, and the following techniques were brought into focus:
(1) Introduction of amino acids (tyrosine (Y), serine (S), and the like) forming a hydrogen bond with a fluorescent chromophore into a position near the chromophore can produce an effect of extending a n-bond of the chromophore, as well as a color change to the longer wavelengths and the varied luminescence intensity (Non-patent Literature 16).
(2) Because the fluorescent chromophore contains a phenyl group in its skeleton, it was possible to improve the optical properties of fluorescent proteins by introducing an amino acid containing a phenyl group as a side chain into a position near the chromophore so as to interact with the phenyl group of the chromophore (Non-patent Literature 17).
(3) A stable and high-intensity fluorescent color was achieved by introducing a hydrophobic amino acid into a position near a fluorescent chromophore so as to hydrophobically interact with the fluorescent chromophore (Non-patent Literature 18).

As described above, it was found that, in regard to the fluorescent protein, there is a case where the introduction of a mutation into an amino acid near a fluorescent protein chromophore effectively improves the fluorescent protein.

The comparison of the substrate of marine luciferases with the fluorescent protein chromophore reveals that their chemical structures are closely similar to each other. Therefore, it was inferred that they shares the same chromophores having very similar chemical structures; and that the fluorescent protein embeds the chromophore inside of the protein, whereas the luciferase recruits the chromophore from the outside of the luciferase as the substrate (FIG. 3C).

Inference 5: In the previous study by the present inventors on luciferase, there is a case of successful improvement of luminescent performance by partially overlapping sequences near the enzyme active site. From this case, improvement in the properties of luciferase can also be expected by a technique of partially overlapping sequences near the enzyme active site.
(2-2) Based on the above five inferences, a strategy for introduction of various mutations by conservative amino acid replacement into 18 positions including a putative luciferase active site and the vicinity thereof was examined (FIG. 4A), and a DNA encoding 57 GLuc variants was prepared in accordance with the strategy (FIG. 4). In the figure, “L74I,” for example, indicates that a DNA mutation that replaces L (Leu) at position 74 with I (Ile) in the amino acid sequence of GLuc was introduced into GLuc gene; and “F89W, I90L” refers to a variant that replaced F (Phe) at position 39 with W (Trp) and, simultaneously, I (Ile) at position 90 with L (Leu).

### Example 3

### Comparison of Bioluminescence Intensities of GLuc into which Amino Acid Mutation has been Introduced

A pcDNA 3.1(+) vector into which the DNA of GLuc variants prepared in Example 2 was inserted was introduced into COS-7 cells cultured in a 12-well plate, followed by incubation for 16 hours.

Subsequently, cell lysates were prepared from the COS-7 cells in each well (5-minute lysis), and a spectrum was measured in the presence of a luciferase-specific substrate (coelenterazine), using a fluorescence spectrophotometer (F-7000, Hitachi), thereby examining the effect of the introduced mutation. FIGS. 4B-1 and 4C show the results. According to FIGS. 4B-1 and 4C, some variants were found to exhibit longer wavelength bioluminescence and remarkably high luminance compared to the original GLuc. Specifically, first, in the case of an introduction of a mutation that potentially induces an interaction between the substrate and a phenyl group, a redshift of about 10 nm was observed with a variant (Y97W) in which tyrosine (Y) at position 97 was mutated to tryptophan (W). From this result, it can be analyzed that a n-bond was extended because a bond was formed between the substrate and the phenyl group.

Further, as a result of a series of introductions of amino acid mutations that form a hydrogen bond with the above substrate, a redshift of about 7 nm in the spectrum was observed when histidine (H) at position 95 and aspartic acid (D) at position 100 were multiply mutated to glutamine (Q) and asparagine (N), respectively. From this result, it can be analyzed that a n-bond was partially extended because a hydrogen bond was formed between the substrate and each amino acid. Further, when F89W/I90L/H95E/Y97W mutations were introduced into the original GLuc, the peak luminescence wavelength of the variant was redshifted up to 35 nm (FIG. 4D). Note that, although it is not shown in FIGS. 4C or 4D, it has been confirmed that I90L variant also causes a shift of 15 nm at maximum to the long wavelength side (FIG. 4B-2).

The above-described variable species is advantageous particularly in terms of the observation of bioluminescence in vivo because the variable species shows long-wavelength bioluminescence. FIG. 4E shows the results of the observations using a 610 nm long-pass filter on bioluminescence of 610 nm or longer (high tissue permeability) emitted by each variant. As predicted, it was found that I90L and the like exhibit a much higher optical intensity compared to the original GLuc.

Also, superluminescent variants of GLuc with enhanced optical intensities through introducing a hydrophobic amino acid into the putative active site of the enzyme were generated. FIG. 4B-1 shows a graph formed using a luminescence spectrophotometer (F-7000, Hitachi) with the peak of each luminescence spectrum as the index. Additionally, in the preparation of cell samples, the samples are immersed in a lysis solution for 5 minutes. This allows preparation of non-uniform samples of cells whose organelles are not lysed, by a quick process. As shown in the figure, GLuc variants having particularly high luminance are obtained when the original GLuc is mutated to be F89W, I90L, I90V, I108V, I112L, I115V, I118L, and/or I118V.

From this result, it was found that the bioluminescence increases its luminescence intensity to a very high level as a result of the introduction of amino acid mutations that hydrophobically interact with the substrate of GLuc. For example, F89W was 9 times, I90L was 14 times, I108V was 6.7 times, and I118L was 4.2 times higher than GLuc in terms of the luminescence intensity. In the case of I90L in particular, one amino acid mutation resulted in bioluminescence 14 times higher than the conventional case. This bioluminescence is 14,000 times higher than that of firefly luciferase (FLUC), which is currently the most used commercially.

Further, transformed COS-7 cells with genes of GLuc variants comprising additional mutation sites were prepared, the optical intensities of which were multiply examined under the same experimental conditions as the above-described studies, including preparation of the cell lysate (FIG. 4B-2). Note that FIG. 4B-2 shows GLuc variants having particularly high luminescence intensity along with the wavelengths that showed maximum luminescence intensity. In this case, a protocol for immersing the lysate samples in lysis solution for 20 minutes was employed (uniformed cell lysates even whose organelles are lysed). The area of the luminescence spectrum measured by a luminescence plate reader was used as the index of the luminescence intensity. According to FIG. 4B-2, particularly high bioluminescence intensity was shown when the original GLuc was mutated to be I90L, H95E, Y97W, F89W/I90L, I90L/I115L, and F89W/I90L/H95E/Y98W. The figure also shows that each of these variants was shifted to a longer wavelength.

The above result confirmed that the bioluminescence increases its intensity to a very high level as a result of the introduction of amino acid mutations that hydrophobically interact with the substrate into the region corresponding to the active center of GLuc. For example, I90L was 7.1 times (hereinafter, based on the area ratio), H95E was 3.0 times, Y97W was 2.1 times, F89W/I90L was 10.2 times, I90L/I115L was 4.9 times, and F89W/I90L/H95E/Y98W was 4.4 times higher than GLuc in terms of the bioluminescence intensity. I90L, in particular, demonstrated bioluminescence that is 7.1 times higher than that of the original GLuc and 10,000 times higher than that of FLuc, as shown in the experimental results in FIG. 4B-2.

Further, in order to visually compare the GLuc variant having high luminance with other luciferases in terms of the luminescence intensity, a substrate (coelenterazine) was added to tubes containing solutions of FLuc, original GLuc, and the GLuc variant having high luminance (FIG. 5). As a result, the demonstration of very strong bioluminescence in GLuc variant (I90L) having high luminance was visually observed.

FIG. 6 shows relative luminescence intensities of the spectra, where the intensities of the GLuc variant (I90L) of the present invention were compared with those of known luciferases (firefly luciferase (FLuc), Gaussia luciferase (GLuc), click beetle luciferase (CBLuc), and the like). This figure also indicates that the luminescence intensity of the GLuc variant is remarkably high.

### Example 4

### Measurement of Stability of Mutant Luciferases

The bioluminescence stability as well as the high bioluminescence intensity is important enzymatic properties expected of the luciferase. Therefore, in order to examine the stability of the bioluminescence of the luciferase variant having high bioluminescence intensity, which was obtained in the present invention, temporal changes in the bioluminescence intensity after the introduction of the substrate were measured (FIG. 7).

First, a gene encoding each GLuc variant was cloned into pcDNA 3.1(+), and the same amount of plasmid was introduced into COS-7 cells. The cells were collected 16 hours after the introduction, lysed in cell lysate, and then transferred to a microplate reader. PBS buffer in which coelenterazine is dissolved was set to a substrate injector of the plate reader. At the measurement, after the substrate was automatically injected into each well, changes in the luminescence intensities were measured every 0.05 seconds. Specifically, the enzyme activities of GLuc itself, F89W, I90L, H95E, and Y97W were measured. As a result, variants such as I90L, H95E, and Y97W showed relatively stable bioluminescence. In contrast, GLuc and F89W showed relatively unstable luminescence intensity (FIG. 7A).

Further, in order to examine changes in the luminescence intensity over a longer period of time, the bioluminescence was observed 10 minutes after I90L, F89W, and I90V were introduced into the substrate, and values relative to the original GLuc were shown (FIG. 7B). As a result, it was observed that while I90L and I90V were stable, F89W and GLuc itself were relatively unstable, and the bioluminescence intensities thereof were decreased by about 60% within 10 minutes after the introduction of the substrate. From this result, it was confirmed that not only high bioluminescence intensity. but also high stability are ensured when isoleucine (I) at position 90 is mutated to leucine (L) or valine (V).

Further, in order to examine the stability of the luminescence intensity, optical properties of each of the variants, I90L, F89W/I90L, and I90L/I115L, were examined using a substrate solution based on modified Mathew's buffer. FIG. 7C shows the results of the observation on temporal changes in the bioluminescence over a short period of time (5 seconds). FIG. 7D shows the results of the observation, after the substrate was introduced stepwise, on temporal changes in the bioluminescence every second at each stage. The results show that each of the variants (I90L, F89W/I90L, and I90L/I115L) demonstrated a very quick response time after the introduction of the substrate, and the bioluminescence intensity was also much higher than that of the original GLuc. These experimental results also indicate that each of these variants shows a very stable and strong bioluminescence.

Note that the composition of the modified Matthew's buffer contains the following components:
100 mM potassium phosphate, 500 mM sodium chloride, 1 mM ethylenediaminetetraacetic acid (EDTA), 0.15 mg/ml porcine gelatin, pH 5.0, 1% Nonyiphenol ethoxylates (Tergitol NP-9) and 1% Antifoam, 25 mM thiourea, and 0.1% DMSO.

### Example 5

### Synthesis of Further Modified Variants

The above synthetic GLuc was synthesized based on the above study on mutation to create a further modified gene (FIG. 8). The I90L variant of the synthetic GLuc shown in FIG. 8A was:
(A) modified to resemble codons of human genes (GC-rich) for optimal expression in human cells, based on the information on the proportion of human codons acquired from GenBank at NCBI; and
(B) restriction enzyme site was added to the middle or end of the gene in order to enhance the availability of the gene as a GLuc reporter. As shown in FIG. 8A, SacII and EcoRV were introduced at base positions 312 and 340, respectively. These sites were found as appropriate dissection sites by the present inventors in their previous study (Non-patent Literature 12), and are the optimal insertion sites for protein insertion. Additionally, BamHI was introduced at the end of the gene in order for this site to be effectively used as a multicloning site upon fusion to DNAs of other proteins.

Based on the findings obtained from the experimental results in Examples 1 to 7, there are four modification sites including I90L for desired GLuc mutations. FIG. 8B shows the preparation of a GLuc enzyme variant (Mon3: F89W/I90L/H95E/Y97W) with the mutations introduced into all four sites, using human-optimized codons. A specific modification strategy for Mon3 is as follows.

(A) First, because GLuc itself has an extracellular secretion signal at the N-terminal, KDEL as an ER localization signal must be linked to the end of the sequence in order to retain GLuc in the cells.

(B) It has been found that the putative enzyme active site of GLuc is located in the center of the enzyme, and that the key sites that are particularly involved in the activity are distributed from F89 to I131.

(C) Amino acids that are particularly important in the enzyme activity and properties of GLuc are indicated by underlines in the sequence.

(D) A restriction enzyme site is preferably placed at the center (enzyme active site) of the DNA sequence encoding GLuc enzyme so as to facilitate easy preparation of a fragmentation site in the enzyme upon construction of a bioluminescent probe at a later date. For this reason, introducing an EcoRV site within the D114 and I115 region is recommended.

(E) Introducing a BamHI site at the end of GLuc for facilitating fusion with other gene sequences is also effective.

Examples of experiments that used Mon3 are shown in FIGS. 4B and 4D. These figures show that Mon3 has an extremely high bioluminescence activity level, and demonstrated a shift to a long wavelength (a long-wavelength shift of 35 nm at maximum).

### Example 6

### Examination of Substrate Selectivity of Each GLuc Variant

The substrate selectivities of the GLuc variants obtained in the present invention were examined (FIG. 9). COS-7 cells were cultured in a 24-well plate. Thereafter, a plasmid encoding each GLuc variant was introduced into the cells, further followed by incubation for 16 hours. Subsequently, 30 μL of cell lysate was added to the cells. Afterward, the luminescence intensity of each variant was measured under various conditions of coexistence of substrates. FIG. 9A shows the results.

The results confirmed that each variant has a substrate selectivity. Specifically, each of the variants, including the original GLuc itself, exhibited strong bioluminescence only in the coexistence of coelenterazine (CTZ), coelenterazine fcp, or coelenterazine ip. On the other hand, strong bioluminescence was not shown in the coexistence of coelenterazine cp, coelenterazine hcp, or coelenterazine 400A.

FIG. 9B shows the typical spectra, and FIG. 9C shows the maximal luminescence intensities according to each substrate. The results show that the spectrum of each of the variants, including the original GLuc itself, is influenced by the type of substrate. Depending on the substrate, some variants showed a shift in the luminescence intensity to a long wavelength, while other showed a shift to a short wavelength. From these results, it was found that the luminescence of each variant can be shifted to a longer or shorter wavelength by changing the substrate.

### Example 7

### Reporter-Gene Assay Carrying a GLuc Variant

In order to prove the usability of the GLuc variants developed in the present invention, a novel reporter gene assay system was constructed by adding the GLuc variants to pG5Luc (Promega), which is a conventional reporter gene assay system (FIG. 10). First, a vector system having an androgen response element (ARE) in the upstream was created, and a gene encoding the original GLuc or GLuc variant of the present invention was linked to the vector system at the downstream by genetic engineering (FIG. 10A), This novel vector was introduced into COS-7 cells, and the difference in the bioluminescence intensities was measured using a luminometer under the conditions of presence and absence of androgen (male hormone) (FIG. 10B).

As a result, the reporter gene assay system carrying the GLuc variant of the present invention indicated a better signal-to-noise ratio than the reporter gene assay system carrying the original GLuc, and also showed an absolute luminescence intensity about 5 times higher than the conventional case (FIG. 10B).

Further, the kinetics of the luminescence intensity was observed using a plate reader under similar experimental conditions (FIG. 10C). Specifically, after COS-7 cells transfected with the above vector were stimulated for 16 hours under conditions of presence and absence of male hormone (androgen), cell lysate was prepared, and a specific substrate (coelenterazine) was added thereto. Then, the difference in the bioluminescence intensity was immediately measured using a plate reader.

In FIG. 10C above, changes in the luminescence intensity can be quantitatively compared by the area from when the substrate was introduced to when a certain period of time (1 second) has passed. The activity level of male hormone being tested can be accurately quantified from the change in the luminescence intensity of the reporter gene (I90L) by subtracting the area defined by the control (GLuc) from the area defined by the reporter gene (I90L) (FIG. 10D). Further, the areas on the luminescence intensity graph in FIG. 10D were compared. As a result, it was confirmed that the transformed cells transfected with pARE-I90L has a larger area (higher bioluminescence intensity) (FIG. 10E).

### Example 8

### Two-Hybrid Assay Carrying a GLuc Variant

In order to demonstrate the advantages of the GLuc variants, an eukaryotic two-hybrid assay system carrying the GLuc variants was constructed, and an experiment to measure the protein-protein interaction was performed (FIG. 11).

In order to perform the experiment, first, the following plasmids were prepared based on pACT, pBIND, and pG5Luc, which are the conventional plasmids for a typical two-hybrid assay: (1) plasmid encoding ER LBD and BIND, (2) plasmid encoding SH2 domain and ACT, and (3) pG5 plasmid carrying the conventional GLuc or the highly luminenscent variant (I90L) of the present invention as a reporter gene.

Living cells into which the above plasmids (1), (2), and (3) were concurrently introduced were stimulated with the solvent itself (0.5% DMSO) and woman sex hormone (estrogen), separately, for 0 hours and 6 hours. Subsequently, the cells were collected, and the bioluminescence intensities were measured using a luminescence spectrophotometer (FIG. 11B). As a result, the cells having the plasmid carrying the GLuc variant of the present invention indicated a superior S/N ratio when stimulated with woman sex hormone. For example, in the case of the plasmid carrying the GLuc variant, the luminescence intensity was raised up to 4-fold by the 6-hour stimulation, compared to the background; however, in the case of the plasmid carrying the conventional enzyme, the increase in the luminescence intensity was not significant.

Further, temporal changes in the luminescence intensity were measured using a plate reader (FIGS. 11C and 11D). As a result, an increase in the very strong bioluminescence was observed in the case of the plasmid carrying the GLuc variant.

Additionally, the difference in the bioluminescence depending on the estrogen concentration was observed (FIG. 12). For this experiment, COS-7 cells were cultured in a 96-well plate. In addition to the genes encoding ERLBD-BIND and SH2-ACT, pG5-GLuc that expresses the original GLuc itself was introduced into the control group.

In contrast, in addition to the genes encoding ERLBD-BIND and SH2-ACT, pG5-I90L that expresses the relevant GLuc variant was introduced into the experimental group. After cultivation for 12 hours, estrogen stimulation was performed, followed by cultivation for an additional 8 hours. Subsequently, lysis buffer was added to the cells, and the luminescence intensity was measured for 2 seconds after the substrate was introduced with the automated program.

As a result, in comparison to the control group, the experimental group showed a significant difference in the luminescence intensity even by 10⁻¹M estrogen stimulation. The degree of luminescence was more remarkable as the concentration of estrogen was higher. As a result, the superior luminescence properties of the relevant GLuc variant led to an improvement in the detection limit.

Next, the living cells into which the above plasmids (1), (2), and (3) were concurrently introduced were stimulated with the solvent itself (0.5% DMSO) or woman sex hormone (estrogen) for 0 hours, 3 hours, 6 hour, and 18 hours. Subsequently, the cells were collected, and the bioluminescence intensities were measured using a luminescence spectrophotometer (FIG. 11E). As a result, the transformed cells having the plasmid carrying one of the GLuc variants of the present invention indicated a superior S/N ratio when stimulated with woman sex hormone. For example, in the case of the plasmid carrying one of the GLuc variants, the luminescence intensity was raised up to 2-fold by the 3-hour stimulation; however, in the case of the plasmid carrying the conventional enzyme, an increase in the luminescence intensity was indistinctive. This result shows that, because the GLuc variant prepared by the present inventors has high luminance, a distinctive increase in strong luminescence was observed even when the stimulation was performed for a significantly reduced period of time (i.e., even when the expression level was low) compared to the conventional case.

### Example 9

### Development of Single-Molecule-Format Bioluminescent Probes Carrying GLuc Variants

In order to further examine the advantages of the highly-luminescent variants of the present invention, a single-molecule-format bioluminescent probe was synthesized based on this variant (FIG. 13). For the synthesis, first, the variant having high luminance (GLuc variant having F89W/I90L mutations) and GLuc variant having four mutations (F89W/I90L/H95E/Y97W) were both dissected into two fragments, and AR LBD and an LXXLL motif were inserted into the space (a probe having the former variant was named 8990N, and a probe having the latter variant was named Mon3N). These probes were inserted into living cells, and the difference in the luminescence intensity was measured under conditions of presence and absence of male hormone. Further, a control probe was prepared by dissecting the original GLuc itself into two fragments in a similar manner and inserting GR LBD and an LXXLL motif into the space (named SimGR3), and the S/N ratio was measured under conditions of presence and absence of stress hormone (cortisol).

FIG. 13A shows the operating principle of this probe. First, a stress hormone receptor recognizes the stress hormone, and causes an alteration in the structure. As a result, the receptor binds to the adjacent LXXLL motif, which thereby causes recombination of the fragments of the dissected luciferase. At this time, the activity of the stress hormone can be measured using the reversed enzyme activity as the index.

The sensitivity of SimGR3, 8990N, and Mon3N prepared for this experiment to the stress hormone was measured (FIG. 13B). As a result, 8990N carrying the GLuc variant of the present invention showed strong bioluminescence, and the sensitivity thereof was not inferior to that of the probe carrying the original GLuc. The luminescence properties of 8990N also showed similar results in the measurement of temporal changes in the luminescence intensity (FIG. 13C). Further, as a result of the measurement of the luminescent values of the conventional SimGR3 and 8990N of the present invention using a 610 nm long-pass filter, 8990N showed luminescence properties superior to those of the original SimGR3. In contrast, in the case of the conventional SimGR3, the difference in the luminescence intensities was not significant enough to distinguish between the presence and absence of stress hormone (FIG. 13D).

### Example 10

### Measurement of Luminescence Activities of MpLuc1 and its Variants

Further, to test the general applicability of the enzyme active site and the mutated sites, which were found in the present invention, on marine luciferases, a group of novel bioluminescence enzyme variants based on Metridia pacifica luciferase (MpLuc1), which is an example of marine animals, was prepared (FIG. 14).

Specifically, the following were synthesized by genetic engineering: original MpLuc1 itself, MpLuc1 with a mutation introduced into Y113 (hereinafter named “Y113F”; this corresponds to F89 of GLuc), MpLuc1 with a mutation introduced into I114 (hereinafter named “I114L”; this corresponds to I90 of GLuc), and a variant transfected with four mutations including I114L of MpLuc1 (this variant carries 4 mutation sites, i.e., Y113W, I114L, H119E, and Y121W, and is named “MpLuc4”; and these mutations correspond to F89, I90, H95, and Y97 or GLuc, respectively). These variants were individually introduced in equal amounts into living cells, and their relative luminescence intensities were measured.

First, the results of the measurement of the luminescence spectrum of each variant show that I114L exhibits a higher luminescence intensity compared to the original MpLuc1. It was also observed that MpLuc4 showed a spectrum shift to a long wavelength, compared to the original MpLuc1 (FIG. 14A). Further, as a result of the examination of the luminescence intensity of the above variant using a 610 nm long-pass filter, the variant was found to show stronger bioluminescence, compared to the original MpLuc1 (FIG. 14B). Still further, temporal changes in the luminescence intensities after the introduction of the substrate were observed using a microplate reader in order to examine the luminescence properties of each variant, and the results showed that each variant demonstrates stable bioluminescence, compared to the original MpLuc1 (FIG. 14C). Unlike other variants, MpLuc4 in particular was found to sustain 60% of its initial luminescence intensity in 10 seconds after the introduction of the substrate.

### Example 11

### Measurement of Luminescence Activities of MLuc and its Variants

To test the general applicability of the enzyme active site and the mutated sites, which were found in the present invention, on marine luciferases, a novel luciferase variant based on Metridia longa luciferase (MLuc), which is an example of marine animals, was prepared (FIG. 15).

Specifically, the following were synthesized by genetic engineering: original MLuc itself, MLuc with I123L mutation introduced (hereinafter named “I123L”; this corresponds to I90 of GLuc), and a variant transfected with four mutations including I123L of MLuc (named “MLuc4”; four mutation sites are Y122W, I123L, H128E, and Y130W, which correspond to F89, I90, H95, and Y97 or GLuc, respectively). These variants were individually introduced in equal amounts into living cells, and their relative luminescence spectra were measured. As a result, it was found that the luminescence intensity of I123L was increased compared to the original MLuc because of the introduction of the mutation (FIG. 15A). It was also found that the position of the maximum luminescence intensity (λ max) also varies due to the introduction of the mutation. In order to examine the luminescence stability of the variants produced in the present invention, temporal changes in the luminescence intensity after the introduction of the substrate were measured every 0.5 seconds (FIG. 15B). As a result, MLuc variant (MLuc4) with mutations introduced into 4 positions showed the most stable bioluminescence. In contrast, the original MLuc was found to lose 90% of the initial luminescence intensity only 10 seconds after the introduction of the substrate. Further, in order to examine the long-wavelength luminescence intensities of the variants, the luminescence intensities of the variants were measured using a 610 nm long-pass filter. The results show that I123L produces strong bioluminescence in the red wavelength. Results similar to the above tendency were obtained from the measurement of the luminescence intensities before and after the introduction of the substrate (FIG. 15D). It was found that the enzyme variants into which the variants were introduced based on the findings of the present invention show bioluminescence properties superior to those of the original type.

### Example 12

### In Vivo Imaging Using GLuc Variants

Taking advantage of the improvements (high luminance, long wavelength shift, stability, and the like) of the GLuc variants, the applicability thereof to the biological system was examined (FIG. 16).

First, pcDNA 3.1 plasmid encoding 8990, which is a GLuc variant having high luminance, was produced and introduced into COS-7 cells. At the same time, as a control, pcDNA 3.1 plasmid encoding the original GLuc itself was similarly introduced into COS-7 cells. Subsequently, both of these transformed cells were transplanted into subcutaneous tissues of the back on the left and right sides of BALE/c nude mice (5-week-old female). After waiting 12 hours to allow stabilization of the transplanted cells, an equal amount of substrate (coelenterazine) was injected. Subsequently, the developed bioluminescence intensities were compared (FIG. 16A). As a result, stronger bioluminescence was observed at the site into which the transformed cells that express 8990 were transplanted.

Further, living cells carrying a mammalian two-hybrid system that expresses the GLuc variant were prepared, and an in vivo imaging system was established using the prepared living cells (FIG. 16B).

First, two plasmids (pBIND and pACT) in which a woman sex hormone receptor (ER LBD) binds to BIND and Src SH2 domain binds to ACT, respectively, were prepared. At the same time, as a signal response system, a plasmid containing a Gal4-responsive element in the upstream and expressing I90L was also prepared (pG5-I90L). As a control, a plasmid that expresses the original GLuc itself was prepared (pG5-GLuc).

The above three plasmids (pBIND, pACT, and pG5-I90L or pG5-GLuc) were co-transfected into COS-7 cells, thereby preparing transformed cells. The transformed cells were transplanted into subcutaneous tissues of the back on the left and right sides of BALE/c nude mice (5-week-old female). At this time, the control cells (pG5-GLuc) were transplanted into the left back side, and the transformed cells carrying pG5-I90L were transplanted into the right back side. A 12-hour waiting time was further allowed for stabilization of the transplanted cells.

Subsequently, woman sex hormone or a solvent (0.1% DMSO) was injected into each mouse, and the luminescence intensity was observed 6 hours after the injection (FIG. 16B). As a result, the mouse stimulated with the woman sex hormone showed stronger bioluminescence. Additionally, in the mouse stimulated with the woman sex hormone, the right back side (expressing I90L) showed stronger bioluminescence than the left side (expressing GLuc). This result can be analyzed as confirming that even when the same amount of GLuc or I90L was expressed by woman sex hormone stimulation, I90L showed stronger bioluminescence because the luminescence of I90L is higher and more stable.

### Example 13

### Illumination of Metastases in Mice

Metastases of murine B16 melanoma to the organs of BALB/c nude mice were illuminated (FIGS. 18 and 19).

Briefly, murine B16 melamona cells (Riken, Japan) were transiently transfected with a pcDNA 3.1 plasmid encoding the original GLuc (pGluc) or a pcDNA 3.1 plasmid encoding I90L (pI90L) using a lipid reagent (Lipofectamine 2000, Invitrogen). Sixteen hours after the transfection, the cells were washed and resuspended in PBS. After the cell counting, an equivalent number of the cells (50 μL, 1×10⁵) was intravenously (iv) implanted into mice via the tail vein. After injection of native CTZ (50 μg per mouse) via the tail vein, the light emission was integrated for two minutes (mice) or five seconds (organs) using IVIS Lumina XR (Xenogen). The optical intensities at the sites of living mice and organs were summarized in FIG. 18.

Bioluminescence intensities of I90L and F89W/I90L/H95E/Y97W (Monsta) in living mice (n=3) were also compared. First, COS-7 cells were transiently transfected with pI90L and pMonsta (a pcDNA 3.1 plasmid encoding F89W/I90L/H95E/Y97W) using Lipofectamine 2000 (Invitrogen). Sixteen hours after the transfection, the cells were washed, harvested, and counted. An equivalent number of the cells (100 μL, 1×10⁵) was sc implanted on the back of mice. After iv injection of CTZ (50 μg per mouse) via the tail vein, the light emission was integrated for one minutes using the IVIS system.

The results showed that almost equivalent amounts of photons were observed from the sites carrying I90L or Monsta as expected from the percents of photons greater than 600 nm (%₆₀₀) (i.e., 8.8±2.5×10⁵ and 7.9±0.5×10⁵ photons/sec/cm²/sr, respectively).

### INDUSTRIAL APPLICABILITY

The measurement performance of assays can be dramatically improved by using luciferase of the present invention in measurements of ligands based on the conventionally widely used reporter gene assay methods, or by using the same in place of bioluminescence enzyme in the conventional bioluminescence probes. Accordingly, the luciferase of the present invention can be widely used in applications such as basic biological studies, and development of diagnostic reagents in medical and pharmaceutical sciences, and analytical chemistry.

Sequence Listing

1. A luciferase variant with improved optical property obtained by replacing at least one amino acid residue among the amino acid sequence of a marine luciferase at positions corresponding to positions 89 to 118 in the amino acid sequence of Gaussia luciferase (GLuc), wherein an amino acid residue at a position corresponding to at least one selected from positions 89, 90, 95, 97, 100, 108, 112, 115, and 118 in the amino acid sequence of GLuc is replaced by way of conservative amino acid replacement.

2. The luciferase variant according to claim 1, wherein the conservative amino acid replacement includes at least one replacement of the following:
(i) replacement of a hydrophobic amino acid residue at a position corresponding to at least one selected from positions 89, 90, 97, 108, 112, 115, and 118 in the amino acid sequence of GLuc with another hydrophobic amino acid residue, and
(ii) replacement of a hydrophilic amino acid residue at a position corresponding to at least one selected from positions 95 and 100 in the amino acid sequence of GLuc that forms a hydrogen bond with another hydrophilic amino acid residue.

3. The luciferase variant according to claim 2, wherein the replacement of a hydrophcbic amino acid residue with another hydrophobic amino acid residue includes at least one replacement of a hydrophobic and nonpolar aliphatic amino acid residue at a position corresponding to at least one selected from position 90, 108, 112, 115, and 118 in the amino acid sequence of GLuc with another hydrophobic nonpolar and aliphatic amino acid residue, and
(ii) replacement of a hydrophobic aromatic amino acid residue at a position corresponding to at least one selected from positions 89 and 97 in the amino acid sequence of GLuc with another hydrophobic aromatic amino acid residue.

4. The luciferase variant according to claim 3, wherein the replacement of a hydrophobic and nonpolar aliphatic amino acid residue with another hydrophobic and nonpolar aliphatic amino acid residue is replacement of isoleucine with an amino acid residue selected from leucine, tryptophan, and valine.

5. The luciferase variant according to claim 3, wherein the replacement of a hydrophobic aromatic amino residue with another hydrophobic aromatic amino residue is replacement of an amino acid residue at a position corresponding to at least one position selected from positions 89 and 97 in the amino acid sequence of GLuc with tryptophan (W).

6. The luciferase variant according to claim 2, wherein the replacement of a hydrophilic amino acid residue at a position corresponding to at least one selected from positions 95 and 100 in the amino acid sequence of GLuc that forms a hydrogen bond with another hydrophilic amino acid residue is at least one replacement selected from replacement of an amino acid residue corresponding to position 95 in the amino acid sequence of GLuc with glutamic acid (E), and replacement of an amino acid residue corresponding to position 100 in the amino acid sequence of GLuc with asparagine (N).

7. A luciferase variant obtained by way of conservative amino acid replacement that includes replacement of an amino acid residue corresponding to position 90 in the amino acid sequence of GLuc among the amino acid sequences of marine luciferase with leucine.

8. The luciferase variant according to claim 7, wherein the conservative amino acid replacement further includes at least one replacement selected from replacement of an amino acid residue corresponding to position 89 in the amino acid sequence of GLuc with tryptophan, and replacement of an amino acid residue corresponding to position 115 in the amino acid sequence of GLuc with leucine.

9. The luciferase variant according to claim 7, wherein the conservative amino acid replacement further includes replacement of an amino acid residue corresponding to position 95 in the amino acid sequence of GLuc with glutamic acid or glutamine, and replacement of an amino acid residue corresponding to position 97 in the amino acid sequence of GLuc with tryptophan.

10. The luciferase variant according claims 1, wherein the marine luciferase is one luciferase selected from Gaussia luciferase (GLuc) and Copepod luciferase (MLuc, MpLuc1, and MpLuc2).

11. A DNA encoding the luciferase variant according to claims 1.

12. An expression vector comprising the DNA according to claim 11.

13. A transformed cell in which the DNA according to claim 11 is introduced in a manner such that the DNA can be expressed.

14. A method for improving optical property of marine luciferase, comprising replacing at least one amino acid residue among the amino acid sequences of a marine luciferase at positions corresponding to positions 89 to 118 in the amino acid sequence of Gaussia luciferase (GLuc), wherein an amino acid residue at a position corresponding to at least one selected from positions 89, 90, 95, 97, 100, 108, 112, 115, and 118 in the amino acid sequence of GLuc is replaced by way of conservative amino acid replacement.

15. A bioluminescent probe with improved optical property that adopts a working mechanism of resurrecting luminescent function by reconstituting dissected luciferase fragments, the probe using the luciferase variant according to claims 1 as the luciferase.

16. A DNA encoding a bioluminescent probe according to claim 15.
